(19) United States
(12) Patent Application Publication
(10) Pub. No.: US 2018/0228815 A1
(43) Pub. Date: Aug. 16, 2018

(54) NARINGENIN AND ASIATIC ACID COMBINATION TREATMENT OF CANCERS

(71) Applicant: The Chinese University of Hong Kong, Hong Kong (CN)

(72) Inventor: HUI YAO LAN, Hong Kong (CN)

(21) Appl. No.: 15/808,344

(22) Filed: Nov. 9, 2017

Related U.S. Application Data
(60) Provisional application No. 62/419,565, filed on Nov. 9, 2016.

(51) Int. Cl. A61K 31/56; A61K 31/352; A61P 35/00

(52) U.S. Cl. CPC A61K 31/56; A61P 35/00; A61K 31/352

(57) ABSTRACT

The present invention resides in the surprising discovery of the previously unrecognized synergistic effects resulted from combined use of narigenin and asiatic acid. New methods and compositions are therefore provided for the treatment or prevention of various types of cancer.

### RELATED APPLICATIONS

This application claims priority to U.S. Provisional Patent Application No. 62/419,565, filed Nov. 9, 2016, the contents of which are incorporated by reference in the entirety for all purposes.

### BACKGROUND OF THE INVENTION

Cancer is an umbrella term for a variety of potentially deadly diseases involving improper and uncontrolled cell proliferation with the potential of spreading or metastasis. In 2012, the latest year global cancer data are available, there were 14.1 million newly diagnosed cancer cases (among which there were 7.4 million cases in men and 6.7 million in women) and 8.2 million cancer deaths worldwide. By 2030, the global numbers are expected to reach 21.7 million new cancer cases and 13 million cancer deaths. The cancer burden on the global population will probably be even larger in the future because of the adoption of western lifestyles, such as smoking, poor diet, physical inactivity, and fewer childbirths, in economically developing countries. The total economic impact of premature death and disability from cancer worldwide was $895 billion in 2008, representing 1.5 percent of the world's gross domestic product (GDP). This economic toll from cancer is nearly 19 percent higher than heart disease, the second leading cause of economic loss. One out of every four deaths in the United States is from cancer. Cancer is second only to heart disease as a cause of death in the US. About 1.2 million Americans are diagnosed with cancer annually; more than 500,000 die of cancer annually.

Because of the great medical prevalence of cancers to human health and the significant impact on the global economy, there remains an urgent need to develop new and effective means for treating and preventing various types of cancers. The present invention addresses this and other related needs.

### BRIEF SUMMARY OF THE INVENTION

Naringenin (NG) and asiatic acid (AA) individually have been known as being involved in different cellular signaling pathways (see, e.g., EP 2162274). Separately, naringenin and asiatic acid have been used to treat cancer and associated diseases (see, e.g., U.S. Patent Application No. 2004/0097463; U.S. Pat. No. 5,145,839; WO 1999/01567; EP 0352147; WO 2001/051043). Together, naringenin and Asiatic acid have been used for treating fibrosis, see, e.g., WO2014/063660. The present inventor made the surprising discovery that, when naringenin and Asiatic acid are administered together, a synergistic effect is achieved in inhibiting cellular proliferation, especially cancer cell proliferation. As such, the present invention provides novel methods and compositions effective for the treatment and prevention of cancers at various anatomic sites. In some embodiments, the present invention is applicable to cancer patients who are not diagnosed with other conditions treatable with naringenin and Asiatic acid co-administration, e.g., fibrosis.

In one aspect, the invention provides a new method of inhibiting cell proliferation. The method includes the step of contacting the cell with an effective amount of naringenin and Asiatic acid. The cell may be a part of a tissue or an organ, such as liver, kidney, skin, or lung. In some embodiments, the contacting step comprises subcutaneous, intramuscular, intravenous, intraperitoneal, topical, or oral administration. In some embodiments, the effective amount of naringenin is about 1-10 mg/kg (e.g., about 2 or 5 mg/kg) body weight at the lower end and about 100 to 500 mg/kg (e.g., about 200 or 250 mg/kg) body weight at the higher end, whereas the effective amount of asiatic acid is about 1-10 mg/kg (e.g., about 2 or 5 mg/kg) body weight at the lower end and about 15-50 mg/kg (e.g., about 20 or 25 mg/kg) body weight at the higher end. In some embodiments, Asiatic acid and naringenin are administered at a weight ratio of about 1:1 or 2 at the higher end to about 1:10 or 15 or 20 at a lower end, for example, the ratio may be about 1:5. In some embodiments, naringenin and Asiatic acid are administered in a single composition. In other embodiments, naringenin and Asiatic acid are administered in two separate compositions. Naringenin and Asiatic acid are administered in any appropriate form, including but not limited to, a solution, a powder, a gel, a cream/paste, a tablet, or a capsule. Generally, the ratio of Asiatic acid to naringenin in weight varies from 1 to 0.001 or less; 0.001; 0.002; 0.005; 0.01; 0.025; 0.05; 0.1; 0.25; 0.50; 0.75; 1.0; 2.0; 2.5; 5.0; 7.5; 10; 20; 25; 50; 75; 100; 200; 250; 300; 400; 500; 600; 700; 800; 900; or 1,000, or even higher.

In another aspect, the invention provides a new composition that comprises (1) an effective amount of naringenin and Asiatic acid and (2) a pharmaceutically acceptable excipient. In some embodiments, Asiatic acid and naringenin are present in the weight ratio of about 1:1 or 2 at one end to about 1:10 or 15 or 20 at the other end, for example, the ratio may be about 1:5. In some embodiments, the composition is formulated for subcutaneous, intramuscular, intravenous, intraperitoneal, topical, or oral administration. For example, the composition may be in the form of a solution, a powder, a gel, a cream, a paste, a tablet, or a capsule. Generally, the ratio of Asiatic acid to naringenin in weight in the composition varies from 1 to 0.001 or less; 0.001; 0.002; 0.005; 0.01; 0.025; 0.05; 0.1; 0.25; 0.50; 0.75; 1.0; 2.0; 2.5; 5.0; 7.5; 10; 20; 25; 50; 75; 100; 200; 250; 300; 400; 500; 600; 700; 800; 900; or 1,000, or even higher.

In yet another aspect, the present invention provides a kit for inhibiting proliferation of cells, especially cancer cells. The kit contains at least two containers: the first container contains a first composition, which comprises narigenin; and the second container contains a second composition, which comprises Asiatic acid. In some embodiments, the first composition is formulated for subcutaneous, intramuscular, intravenous, intraperitoneal, topical, or oral administration. In some embodiments, the second composition is formulated for subcutaneous, intramuscular, intravenous, intraperitoneal, topical, or oral administration. In some embodiments, the kit further includes an instruction manual for administration of the first and second compositions. Generally, when used together in practicing the present invention, Asiatic acid to naringenin ratio in weight may vary from 1 to 0.001 or less; 0.001; 0.002; 0.005; 0.01; 0.025; 0.05; 0.1; 0.25; 0.50; 0.75; 1.0; 2.0; 2.5; 5.0; 7.5; 10; 20; 25; 50; 75; 100; 200; 250; 300; 400; 500; 600; 700; 800; 900; or 1,000, or even higher.

In each of the above described aspects, the synergistic therapeutic effects of narigenin and Asiatic acid are achievable by substituting Asiatic acid with either of its two known analogs, madecassic acid and asiaticoside, in combined use with narigenin.

### BRIEF DESCRIPTION OF THE DRAWINGS

FIG. 1. Determination of a safe dosage of AA or NG in normal mice. Groups of 4 normal mice were treated with different dosages of asiatic acid (AA) or naringenin (NG) for two weeks and various parameters were measured to determine a safe dosage for the use as therapeutic dose in cancer-bearing mice. As indicated by a highlighted box, AA at 10 mg/kg and NG at 50 mg/kg was selected. Each bar represents the mean±SEM for groups of 4 mice. * p<0.05, ** p<0.01 compared to untreated mice (BLK)

FIG. 2. Determination of the effective ratio between AA or NG on tumor angiogenesis in vitro. Tumor cells (B16F10) were cultured with TGF-beta1 (2 ng/ml) and VEGF mRNA expression by tumor cells was measured by real-time PCR. Results show that addition of AA at luM is able to significantly inhibit TGF-beta1-induced angiogenesis, which is enhanced by addition of NG from 0.5 to 250 uM. Each bar represents the mean±SEM for 3 independent experiments. * p<0.05, ** p<0.01 compared to TGF-beta 1-treated tumor cells.

FIGS. 3A-3B. Combination therapy of AA and NG produces a better therapeutic effect in rebalancing Smad signaling in both B16F10 melanoma and LLC lung cancer mouse models. Western blot analysis shows that combination with AA and NG results in a further reduction of p-Smad3 but increasing Smad7 in both melanoma and lung cancer mouse models. Each bar represents the mean±SEM for groups of six to eight mice. * p<0.05, ** p<0.01 compared to untreated tumors (Ctrl).

FIGS. 4A-4C. Combination therapy of AA and NG produces a better therapeutic effect on cancer growth both B16F10 melanoma and LLC lung carcinoma mouse models. (FIG. 4A) Effect of AA, NG, and AA+NG (CB) on B16F10 melanoma growth. (FIG. 4B) Effect of AA, NG, AA+NG (CB) on LLC lung carcinoma growth. (FIG. 4C) Bioluminescence live imaging analysis of therapeutic effect of AA, NG, AA+NG (CB) treatment on LLC lung carcinoma. Each bar represents the mean±SEM for groups of six to eight mice. * p<0.05, ** p<0.01, *** p<0.001 compared to untreated tumors (Ctrl); # p<0.05, ## p<0.01 compared to individual treatment as indicated.

FIG. 5. AA, NG, and combination therapy of AA and NG do not cause systemic toxicity including peripheral white blood cell counts, serum LDH, liver functions (AST and ALT), and renal function (creatinine) on B16F10 melanoma-bearing mice. Each bar represents the mean±SEM for groups of 6 mice.

FIG. 6. Combination therapy of AA and NG produces a better inhibitory effect on LLC lung carcinoma by largely promoting NK1.1+NKp46+ cells (matured NK cells) in tumor microenvironments. Each bar represents the mean±SEM for groups of 6 mice. *** p<0.001 compared to untreated tumors (Ctrl); ### p<0.001 compared to individual treatment as indicated.

FIG. 7. Combination therapy of AA and NG produces a better inhibitory effect on LLC lung carcinoma by largely promoting NK cell cytotoxicity against tumor in tumor microenvironments. (A) Granzyme B and (B) IFN-γ producing NK cells and concentrations are largely increased in AA+NG treated tumor microenvironments when compared to the use of AA or NG alone. Each bar represents the mean±SEM for 6 mice. ** p<0.01, *** p<0.001 compared to untreated tumors (Ctrl); ## p<0.01, ### p<0.001 compared to individual treatment as indicated.

FIG. 8. Combination therapy of AA and NG produces a better inhibitory effect on LLC lung carcinoma proliferation by largely reducing Ki67+ cells, in vitro tumor colony formation, CD31+ angiogenesis, and vascular endothelial growth factor (VEGF)-producing cells in LLC invasive lung carcinoma. Data represent for groups of 6 mice or at least three-independent experiments

FIG. 9. Combination therapy of AA and NG produces a better inhibitory effect on the invasiveness of LLC lung carcinoma in vivo. Noted that the combined AA and NG therapy blocks the invasive activities of tumor such as MMp2, M9, and MMP13 of LLC lung tumor. Each bar represents the mean±SEM for groups of 6 mice. * p<0.05, ** p<0.01 compared to untreated tumors (Ctrl); # p<0.05 compared to NG treatment as indicated.

FIGS. 10A-10B. Combination therapy of AA and NG produces a better inhibitory effect on B16F10 melanoma invasiveness in vitro. (FIG. 10A) Wound healing assay and (FIG. 10B) transwell assay for the inhibitory effect of AA, NG and AA+NG (CB) on B16F10 melanoma invasion. Each bar represents the mean±SEM for three-independent assays. * p<0.05, ** p<0.01, *** p<0.001 compared to untreated tumors (Ctrl); # p<0.05, ## p<0.01, ### p<0.001 compared to individual treatment as indicated.

FIGS. 11A-11J. Combination therapy with AA and NG produces a better inhibitory effect on tumor growth compared to monotherapy. (FIGS. 11A-11D) Dose-dependent inhibitory effect of AA or NG on B16F10 melanoma volume and weight. (FIGS. 11E, 11F) Combination therapy with an optimal AA (10 mg/kg/day) and NG (50 mg/kg/day) produces a better inhibitory effect on B16F10 melanoma growth when compared to monotherapy. (FIGS. 11G-11J) Combination therapy with an optimal AA (10 mg/kg/day) and NG (50 mg/kg/day) produces a better inhibitory effect on LLC lung carinoma when compared to monotherapy as determined by tumor volume, tumor weight and bioluminescence imaging. Each error bar represents the mean±SEM for groups of six to eight mice. * p<0.05, ** p<0.01, *** p<0.001 compared to Ctrl; # p<0.05, ## p<0.01, ### p<0.001 as indicated.

FIGS. 12A-12B. Combination therapy with AA and NG largely enhances cytotoxic NK cells (NK1.1+NKp46+) infiltrating the LLC tumor microenvironment. (FIG. 12A) Two-color immunofluorescence shows that numbers of cytotoxic NK cells (NK1.1+NKp46+) in LLC tumor microenvironment are largely increased in mice with the combination of AA and NG when compared to each single treatment. NK1.1 (green), NKp46 (red), DAPI (blue). (FIG. 12B) Two-color flow cytometry detects that numbers of cytotoxic NK cells (NK1.1+NKp46+) in peripheral blood of LLC tumor-bearing mice are largely increased in mice with the combination of AA and NG when compared to each single treatment. Each bar represents the mean±SEM for groups of three mice. ** p<0.01, *** p<0.001 compared to Ctrl; ## p<0.01, ### p<0.001 as indicated. Scale bar, 100 μm.

FIGS. 13A-13G. Combination of AA and NG enhances the production of IFN-γ and granzyme B by NK cells both in vivo and in vitro. (FIGS. 13A, 13B) Two-color immunofluorescence detects NK1.1+IFN-γ+ and NK1.1+ granzyme B+ NK cells infiltrating the LLC tumor microenvironment. NK1.1+ cells (green), IFN-γ+ or granzyme B+ cells (red), DAPI (blue). Each error bar represents the mean±SEM for groups of three to four mice. * p<0.05, ** p<0.01, *** p<0.001 compared to Ctrl; ## p<0.01, ### p<0.001 as indicated. Scale bar, 100 μm. (FIGS. 13C, 13D) ELISA shows IFN-γ and granzyme B levels in homogenized LLC tissue. (FIGS. 13E, 13F) Effect of AA (10 μmol), NG (100 μmol), and their combination on levels of IFN-γ and granzyme B in supernatant of cultured splenic NK cells with TGF-β1 (5 ng/ml) detected by ELISA. (FIG. 13G) NK cell cytotoxicity assay with LLC as target cells at E:T ratio of 5:1, 10:1 and 20:1. Each error bar represents the mean±SEM for groups of three independent experiments. * p<0.05, ** p<0.01, *** p<0.001 compared to TGF-β1; # p<0.05, ## p<0.01, ### p<0.001 as indicated.

FIGS. 14A-14C. Combination of AA and NG produces a better outcome in rebalancing Smad signaling in NK cells by inactivating Smad3 while upregulating Smad7. (FIGS. 14A, 14B) Two-color immunofluorescence detecting NK1.1+ p-Smad3+ and NK1.1+ Smad7+ NK cells in LLC tumor microenvironment. NK1.1+ cells (green), p-Smad3+ or Smad7+ cells (red), DAPI (blue). Each bar represents the mean±SEM for groups of three to four mice. ** p<0.1, *** p<0.01 compared to Ctrl; # p<0.5, ### p<0.01 as indicated. Scale bar, 100 μM. (FIG. 14C) Western blot analysis shows p-Smad3 and Smad7 protein expression in response to TGF-β1 (5 ng/ml), AA (10 μmol), NG (100 μmol), and their combination on bone marrow-derived NK cells. Each bar represents the mean±SEM for groups of three independent experiments; * p<0.05, ** p<0.01, *** p<0.001 compared to TGF-β1; # p<0.05, ## p<0.01 as indicated.

FIGS. 15A-15B. Combination of AA and NG attenuates TGF-β1 induced inhibition on NK cell differentiation. (FIG. 15A) Flow cytometry shows inhibitory effect of TGF-β1 on NK cell differentiation as determined by NK1.1+ CD122+ populations in bone marrow-derived NK cells (9 days). *** p<0.01 compared to Ctrl; # p<0.5 as indicated. (FIG. 15B) Flow cytometry detects that the effect of AA, NG, and their combination on bone marrow-derived NK differentiation in response to TGF-β1 (5 ng/ml). Each error bar represents the mean±SEM for groups of three independent experiments. *** p<0.001 compared to TGF-β1; ### p<0.001 as indicated.

FIGS. 16A-16D. Combination therapy with AA and NG reverses the suppressive effect of TGF-β1 on Id2 and IRF2 expression. (A, B) mRNA levels of Id2 and IRF2 in peripheral blood NK cells isolated from LLC bearing-mice detected by real-time PCR; ** p<0.01 compared to Ctrl; ## p<0.01 as indicated. (C, D) mRNA levels of Id2 and IRF2 in AA and NG treated bone marrow-derived NK cells detected by real-time PCR. (E) Id2 and IRF2 expression in AA and NG treated bone marrow-derived NK cells measured by western blotting. Each bar represents the mean±SEM for groups of three to four mice or groups of three independent experiments. ** p<0.01, *** p<0.001 compared to TGF-β1; ## p<0.01, ### p<0.001 as indicated.

FIGS. 17A-17D. Disrupted Id2 or IRF2 impairs the protective effect of AA and NG on NK cell differentiation and maturation in vitro. (FIG. 17A) NK1.1+NKp46+ cells by two-color flow cytometry. (FIG. 17B) CD11b+ NK cells by flow cytometry. (FIG. 17C) Quantitation of NK1.1+NKp46+ cells. (FIG. 17D) Quantitation of CD11b+ cells. Bone marrow-derived NK cells were transfected with si-Id2 or si-IRF2 and then cultured with AA (10 μmol) and NG (100 μmol) under TGF-β1 (5 ng/ml) conditions for 6 days and collected for flow cytometry analysis. Each bar represents the mean±SEM for three independent experiments. *p<0.05, ***p<0.001 compared to non-treated group, # p<0.05, ### p<0.001 as indicated.

FIGS. 18A-18D. Smad3 directly interacts with Id2 and IRF2 gene as a trancriptional repressor in NK cells. (FIG. 18A) Predicted Smad binding sites on Id2 3′UTR. (FIG. 18B) Predicted Smad binding sites on IRF2 3′ and 5′UTR. (FIGS. 18C, 18D) ChIP assay shows that addition of TGF-β1 (5 ng/ml) induces Smad3 directly binding to 3′UTR of both Id2 and IRF2 gene on bone marrow-derived NK cells.

FIG. 19. Combination therapy does not induce severe side effect on LLC bearing mice. (A) White blood cell count performed with peripheral blood collected from LLC bearing mice 27 days after tumor inoculation. Serum isolated from LLC bearing for detection of (B) creatinine (C) LDH (D) AST and (E) ALT. Each error bar represents the mean±SEM for groups of three to four mice.

FIGS. 20a-20B. Combination therapy does not significantly influence NK cell proliferation and NK cell infiltration. (FIG. 20A) MTT assay with bon marrow-derived NK cells treated with AA, NG; and combination therapy. (FIG. 20B) Real-time PCR detecting mRNA level of CXCR3, a chemokine receptor critical for NK infiltration on tumor microenvironment. Each error bar represents the mean±SEM for groups of three independent experiments.

FIG. 21. Knocking down Id2 and IRF2 in bone marrow-derived NK cells. Protein level of Id2 and IRF2 in bone marrow-derived NK cells treated with specific siRNA sequence for Id2 and IRF2 measured by western blot. Each error bar represents the mean±SEM for groups of three independent experiments. *p<0.1, ***p<0.01 compared to TGF-β1, ### p<0.01 compared with CB.

### DEFINITIONS

The term “inhibiting” or “inhibition,” as used herein, refers to any detectable negative or suppressing effect on a target biological or pathological process, such as the proliferation of cancer cells or progression of a cancer. Typically, an inhibition is reflected in a decrease of at least 10%, 20%, 30%, 40%, or 50% in a feature characteristic of the target process (e.g., the rate of cancer cell proliferation) when compared to a control.

The term “cancer” encompasses any disease that involves improper and uncontrolled cellular proliferation resulting in the accumulation or growth of these improperly proliferating cells at an original anatomic site with a tendency of spreading to another anatomic site, a process called metastasis. Cancers can arise from virtually any tissue type and can affect all organs and body parts, some high incidence cancers including lung cancer, colon and rectum cancer, breast cancer, prostate cancer, pancreatic cancer, brain cancer, stomach cancer, liver cancer, esophagus cancer, bladder cancer, kidney cancer, skin cancer (melanoma), and various blood cancers such as leukemia, lymphoma, and myeloma. Humans of all ages can develop cancer.

The term “effective amount,” as used herein, refers to an amount of a substance that produces therapeutic effects for which the substance is administered. The effects include the prevention, correction, or inhibition of progression of the symptoms of a disease or condition and related complications to any detectable extent. In a case where two or more substances are used for a desired effect, the “effective amount” may be expressed in more than one way. For example, the “effective amount” may be expressed in the total amount of all active ingredients, or expressed in a separate amount for each active ingredient, or expressed in a ratio (e.g., in weight or volume ratio) of one ingredient over another. The exact amount of an “effective amount” will depend on the purpose of the treatment as well as the form and identify of the active substance, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

“Naringenin” is a flavanone with the systematic (IUPAC) name 5,7-dihydroxy-2-(4-hydroxyphenyl)chroman-4-one and synonym 4′,5,7-trihydroxyflavanone (CAS Number 480-41-1). Its chemical formula is C₁₅H₁₂O₅ and molecular weight is 272.257. In nature, naringenin is present most abundantly in grapefruits, oranges and tomato skin. High purity naringenin can be purchased from various commercial venders.

“Asiatic acid” is derived from an ancient, traditional herbal extract of the plant Centella asiatica, commonly called Gotu Kola. It is also known as dammarolic acid (CAS Number 464-92-6). Its chemical formula (Hill Notation) is C₃₀H₄₈O₅, and molecular weight is 488.70. Asiatic acid shares many similarities with its analogs, madecassic acid and asiaticoside. It is available through commercial suppliers such as Sigma-Aldrich.

As used herein, the term “administration” encompasses any means of delivering a substance, e.g., an agent with therapeutic or prophylactic effects, to a subject, which may include but is not limited to, systemic, regional, and local applications. Examples of “administration” are injection (such as by subcutaneous, intramuscular, intravenous, or intraperitoneal means), oral ingestion, intake through the nasal cavity or through the eyes or ears, inhalation, transdermal delivery, and anal or virginal deposit, etc.

The terms “pharmaceutically acceptable excipient” and “physiologically acceptable excipient” may be used interchangeably to refer to an inert substance that is included in the formulation of a composition containing an active ingredient to achieve certain characteristics, such as more desirable pH, solubility, stability, bioavailability, texture, consistency, appearance, flavor/taste, viscosity, etc., but in itself does not negatively impact the intended therapeutic or prophylactic effects of the active ingredient.

The term “tissue,” as used herein, refers to an ensemble of cells that are similar in their biological attributes, such as morphology and biological activity, and are from the same origin, such that these cells together carry out a specific function. An “organ” is a collection of different tissues joined in a structural unit to serve a common function.

The term “about,” as used herein, describes a range of plus or minus 10% from a recited value. For example, a value of “about 10” can be any value within the range of 10±1, i.e., between 9 to 11.

### DETAILED DESCRIPTION OF THE INVENTION

### I. Introduction

Cancer is one of the leading cause for human deaths, yet the current treatment for cancer using cytotoxic drugs remains non-specific and ineffective with severe side-effects. In this disclosure, newly developed therapeutic compositions are described for effectively treating cancer by promoting the host immunity against tumor. It was discovered that a compound containing a component purified from the citric fruits called naringenin (NG) and an asiatic acid (AA) from a herb can produce more effective inhibition of tumor growth and invasiveness in two syngeneic mouse models of B16F10 melanoma and LLC invasive lung carcinoma, without causing toxicity to the normal body when compared to the individual use. It was also discovered that enhanced the NK cell development and NK cell-dependent cancer-killing activities are the major therapeutic mechanism of this invention, although the combination therapy of AA and NG can also effectively inhibit tumor growth/proliferation and invasive/migration activities including angiogenesis (CD31⁺) and the expression of matrix metalloproteinases (MMPs). Thus, this invention provides a new emerging and promising meaning for cancer therapy.

This invention resides in the discovery that a mixed composition comprising naringenin and asiatic acid can produce more effective growth inhibition on cancer cells such as B6F10 melanoma and LLC invasive lung cancer cells while exhibiting little to no toxicity to the host body. It has also been revealed that the mixed composition can function as an immune regulator, which promotes NK cell development and NK cell-dependent cancer-killing activities in the cancer microenvironment. Thus, this invention is advantageous over the current anti-cancer therapies, which use cytotoxic drugs and often produce many severe side-effects.

### II. Pharmaceutical Compositions and Administration

The present invention provides pharmaceutical compositions or physiological compositions comprising an effective amount of naringenin and Asiatic acid, which is effective for inhibiting undesirable proliferation of cells, especially cancer cells, in a tissue or organ in both prophylactic and therapeutic applications. Such pharmaceutical or physiological compositions also include one or more pharmaceutically or physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The pharmaceutical compositions of the present invention can be administered by various routes, e.g., oral, topical, subcutaneous, transdermal, intramuscular, intravenous, intranasal, or intraperitoneal. The preferred routes of administering the pharmaceutical compositions are local delivery to an organ or tissue suffering from or at risk of developing cancer (e.g., intraperitoneal injection to an organ) at daily doses of about 0.35-17.5 g, preferably 2.5-5.5 g, of naringenin and about 0.1-3.5 g, preferably 0.5-1.0 g, of Asiatic acid for a 70 kg adult human per day. The appropriate dose may be administered in a single daily dose or as divided doses presented at appropriate intervals, for example as two, three, four, or more subdoses per day.

For preparing pharmaceutical compositions containing naringenin or Asiatic acid, or containing both, one or more inert and pharmaceutically acceptable carriers are used. The pharmaceutical carrier can be either solid or liquid. Solid form preparations include, for example, powders, gels/creams/pastes, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., naringenin and/or Asiatic acid. In tablets, the active ingredient (naringenin and/or Asiatic acid) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient of naringenin and/or Asiatic acid. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active ingredient of naringenin and/or Asiatic acid with encapsulating material as a carrier providing a capsule in which the active ingredient (with or without other carriers) is surrounded by the carrier, such that the carrier is thus in association with the active ingredient. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., naringenin and/or Asiatic acid) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component, such as naringenin and/or Asiatic acid, in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

In addition, the pharmaceutical compositions containing the active ingredient of naringenin and/or Asiatic acid can be administered topically to a patient in the case of treatment or prevention of skin cancer (melanoma). The compositions may be formulated as a gel, a cream, a paste, a powder, or a spray for the ease of use.

The pharmaceutical compositions containing the active ingredient of naringenin and/or Asiatic acid can be administered for prophylactic and/or therapeutic treatments. In therapeutic applications, compositions are administered to a patient already suffering from a condition involving undesirable cellular proliferation such as cancer in an amount sufficient to prevent, cure, reverse, or at least partially slow or arrest the symptoms of the cancer and its complications. An amount adequate to accomplish this is defined as a “therapeutically effective dose.” Amounts effective for this use will depend on the severity of the disease or condition and the weight and general state of the patient, but generally range from about 0.35 g to about 17.5 g of naringenin and 0.1 g to about 3.5 g of Asiatic acid per day for a 70 kg patient, with dosages of from about 2.5 g to about 5.5 g of naringenin and 0.5 g to about 1.0 g of Asiatic acid per day for a 70 kg patient being more commonly used.

In prophylactic applications, pharmaceutical compositions containing naringenin and/or Asiatic acid are administered to a patient susceptible to or otherwise at risk of developing a cancerous disease or condition, in an amount sufficient to delay or prevent the onset of the cancer-related symptoms. Such an amount is defined to be a “prophylactically effective dose.” In this use, the precise amounts of naringenin and Asiatic acid again depend on the patient's state of health and weight, but generally range from about 0.35 g to about 5.5 g of naringenin and 0.1 g to about 3.5 g of Asiatic acid for a 70 kg patient per day, more commonly from about 2.5 g to about 5.5 g of narigenin and 0.5 g to about 1.0 g of Asiatic acid for a 70 kg patient per day.

Single or multiple administrations of the compositions can be carried out with dose levels and pattern being selected by the treating physician. In any event, the pharmaceutical formulations should provide a quantity of naringenin and/or Asiatic acid sufficient to effectively inhibit undesirable proliferation of cells (especially malignant cells) in the patient, either therapeutically or prophylactically.

### III. Kits

The invention also provides kits for inhibiting cellular proliferation, especially undesirable proliferation such as cancer cell proliferation, according to the method of the present invention. The kits typically contain two containers: the first container contains a composition that comprises narigenin and the second container contains a composition that comprises Asiatic acid.

Alternatively, the kits may include a container that contains a pharmaceutical composition having an effective amount of naringenin and Asiatic acid (such as a composition described herein in detail), as well as informational material containing instructions on how to dispense the pharmaceutical composition, including description of the type of patients who may be treated (e.g., patients who are suffering from or are at risk of developing a hyperproliferative disease such as cancer), the schedule (e.g., dose and frequency) and route of administration, and the like.

### EXAMPLES

The following examples are provided by way of illustration only and not by way of limitation. Those of skill in the art will readily recognize a variety of non-critical parameters that could be changed or modified to yield essentially the same or similar results.

### Example 1 Naringenin/Asiatic Acid Inhibition of Cancer Cell Proliferation and Invasiveness

The current anti-cancer drugs are largely cytotoxic with severe side-effect on the body. It has been shown that naringenin can inhibit TGF-β signaling and has anti-cancer activities by inhibiting the tumor cell growth through the S-G2M-dependent mechanism (Patent No: EP2163247 B1; Abaza M S et al: Cancer Cell Int. 2015; 15:46). It is also reported that Asiatic acid has a wide arrange effect on inflammation, fibrosis, and cancer (Patent EP 1313462 B1; US 20040097463 A1; CN 102391351 B; CN 103467560 A). The inventor's group has also previous shown that naringenin (NG) is a Smad3 inhibitor while Asiatic acid (AA) functions as a Smad7 agonist and the combination of both produces a better inhibitory effect on rebalancing TGF-β/Smad3 signaling and has better therapeutic effect on renal fibrosis (Meng et al: Oncotarget 2015; 6:36984, Patent CN 104736151/WO2014063660A1). It was tested whether a compound containing naringenin and asiatic acid can be used for a better treatment for cancer. Unexpectedly, it was discovered that the combined therapy with AA and NG at a ratio 1:5 produces a better anticancer effect when compared to individual one. We also identified that the therapeutic target of this compound is associated with a large promotion of NK cell development and NK cell immunity against cancer without toxicity. Thus, this compound may function as a new immune regulator and suppresses cancer by promoting the host immunity against cancer, which is largely different from the current cytotoxic drugs.

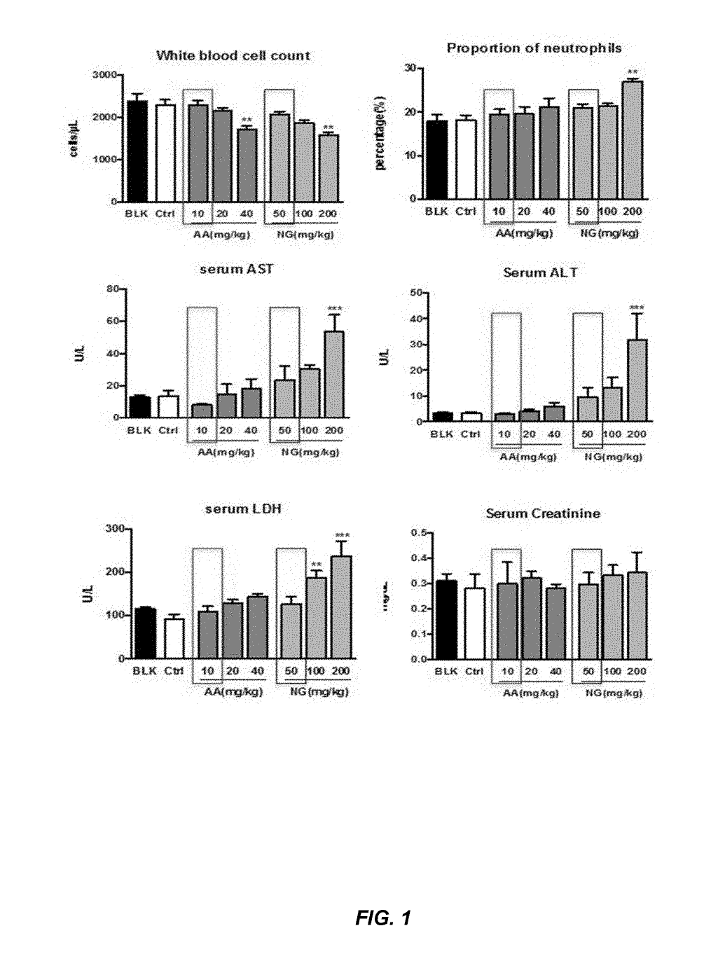

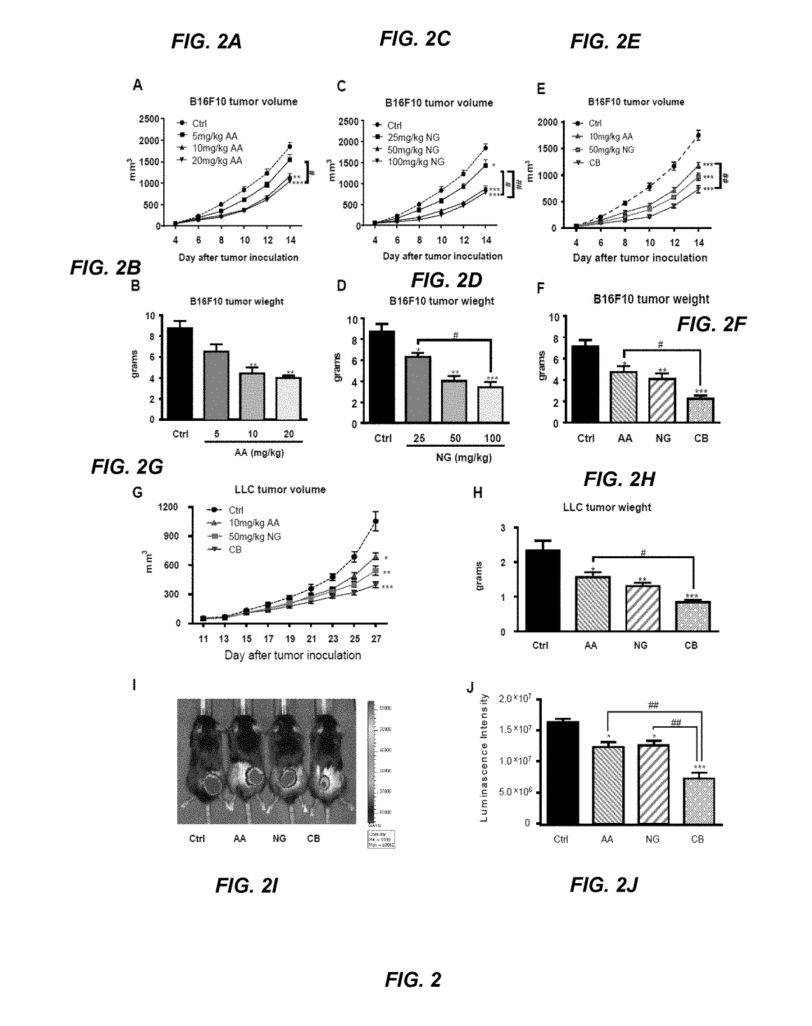

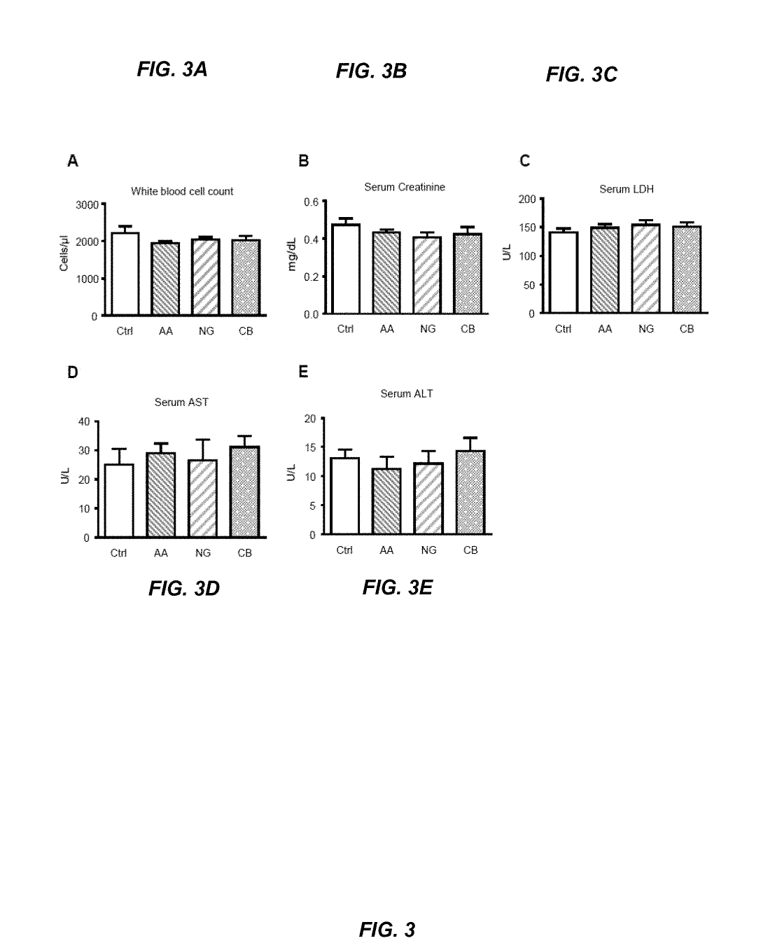

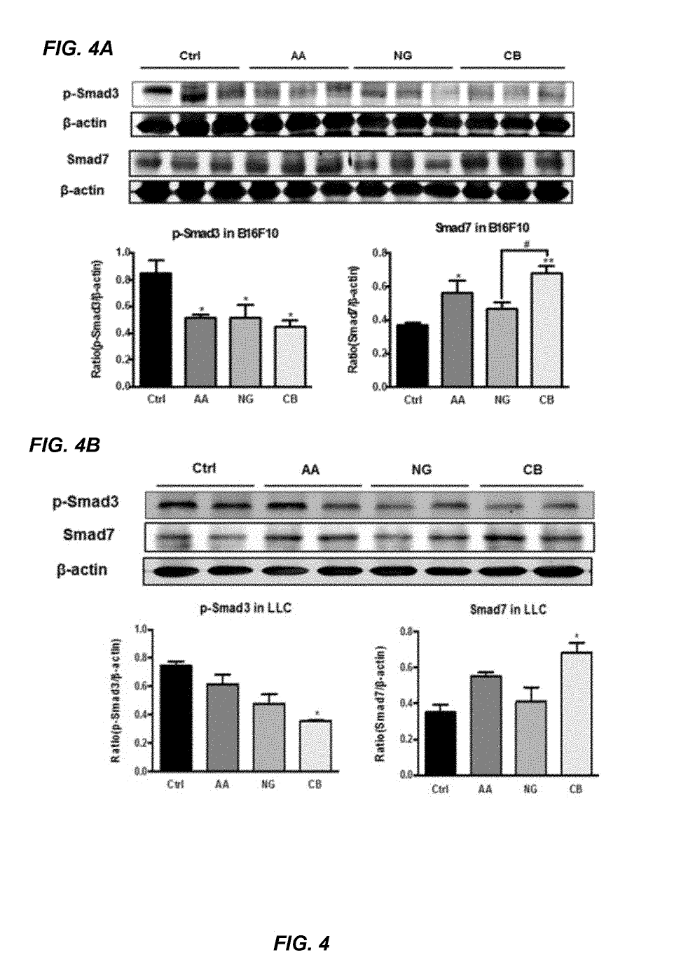

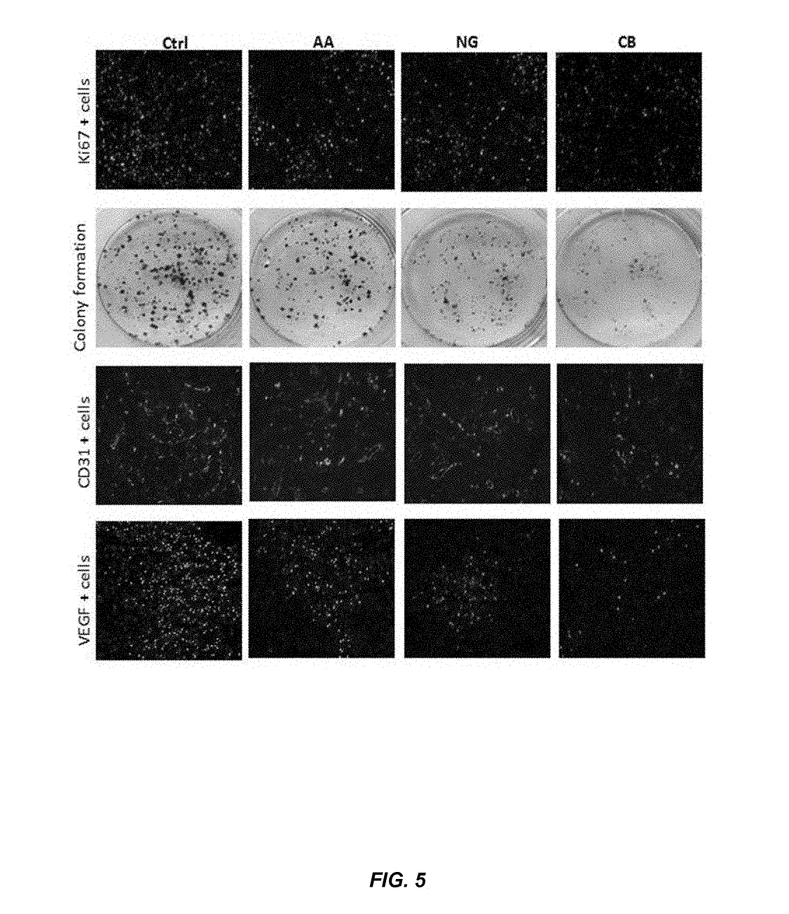

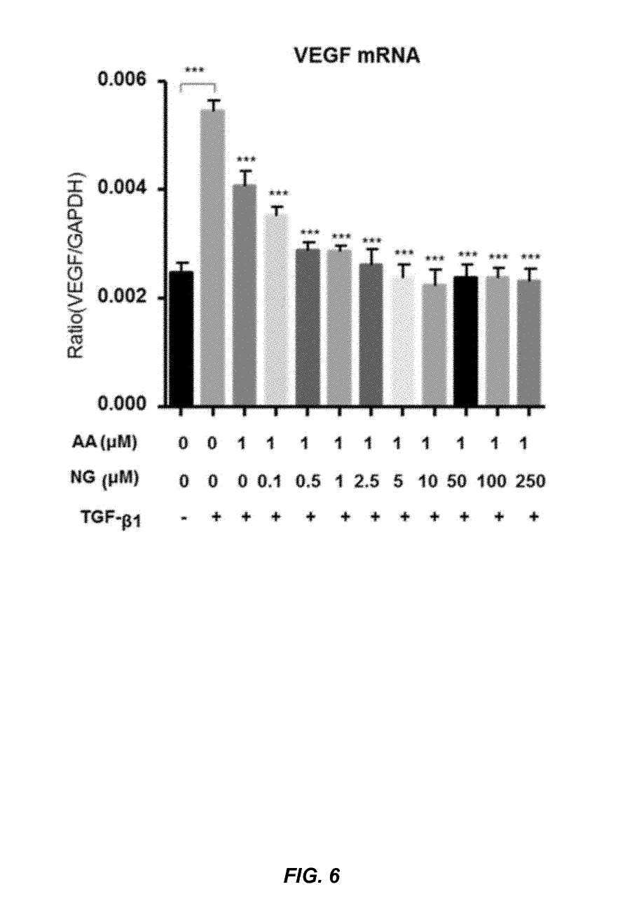

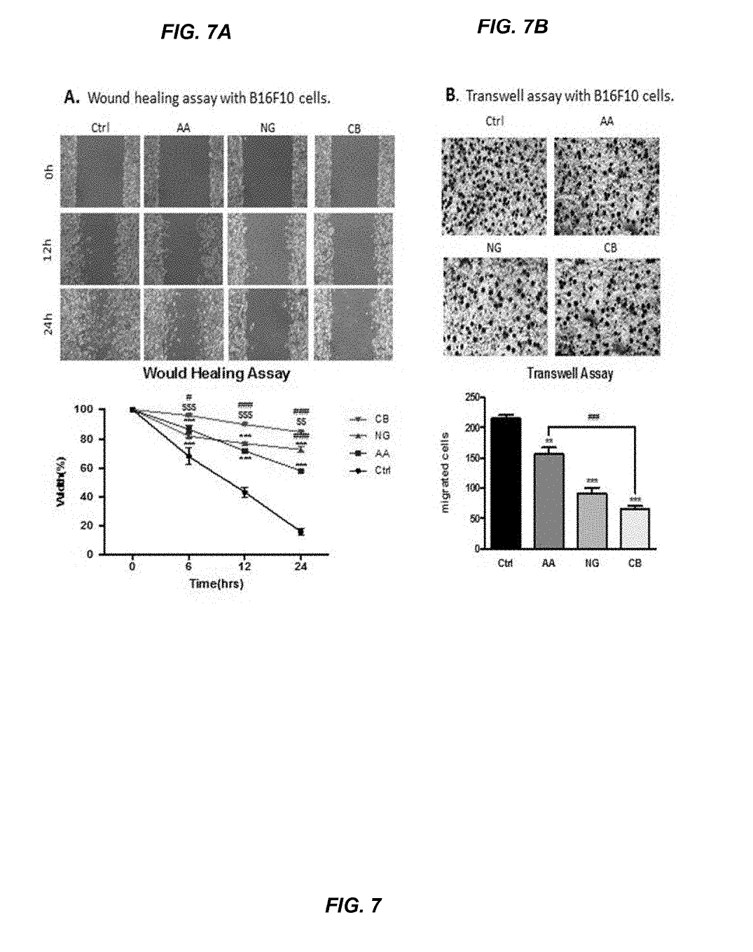

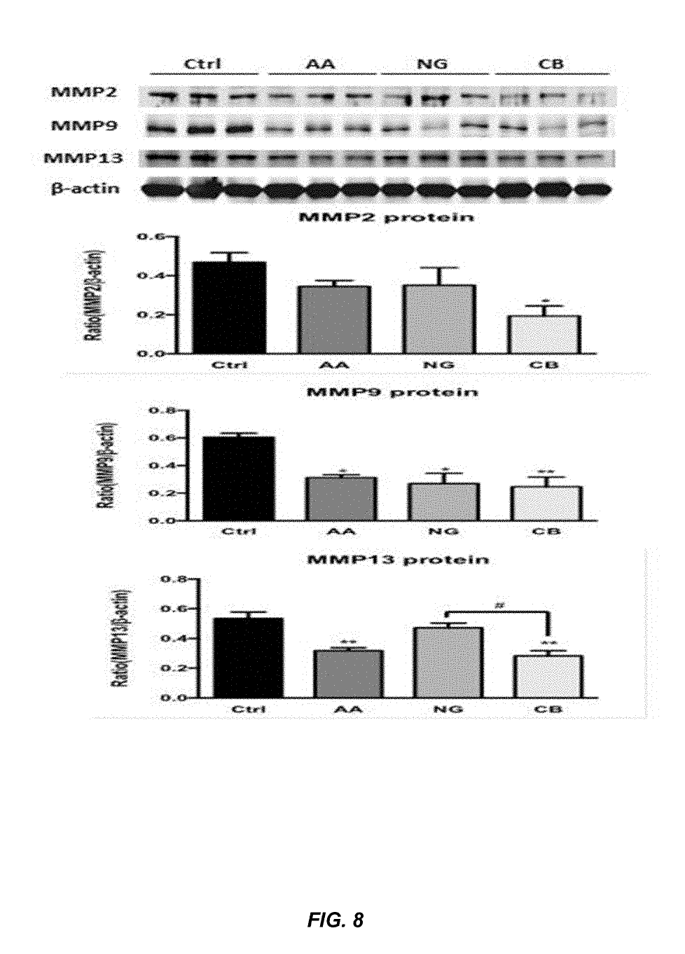

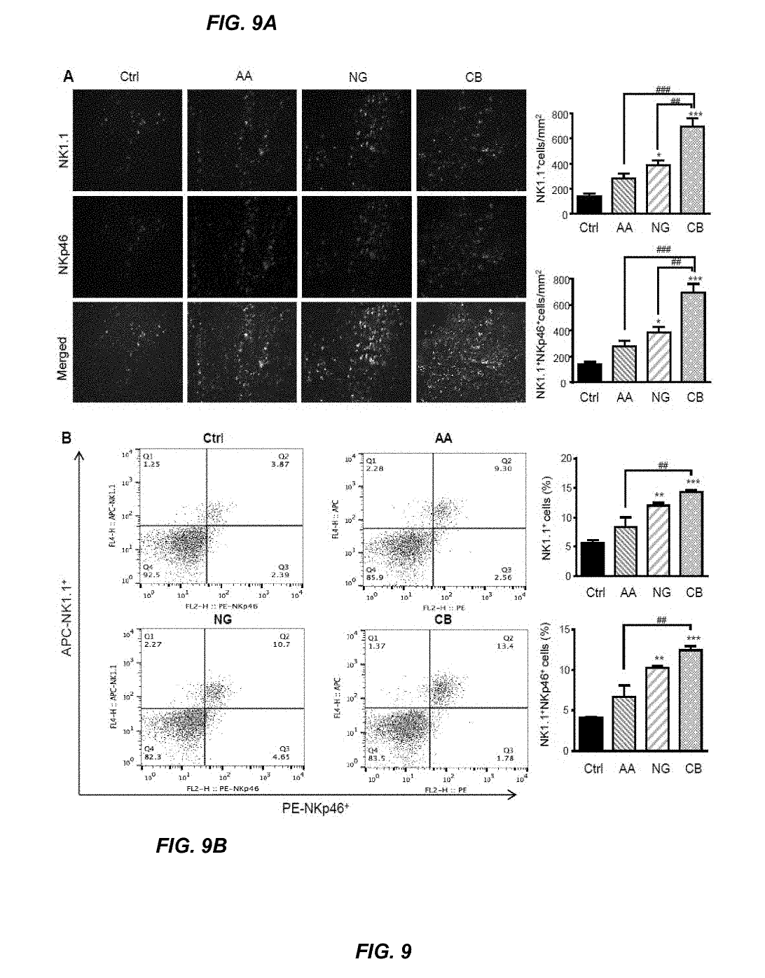

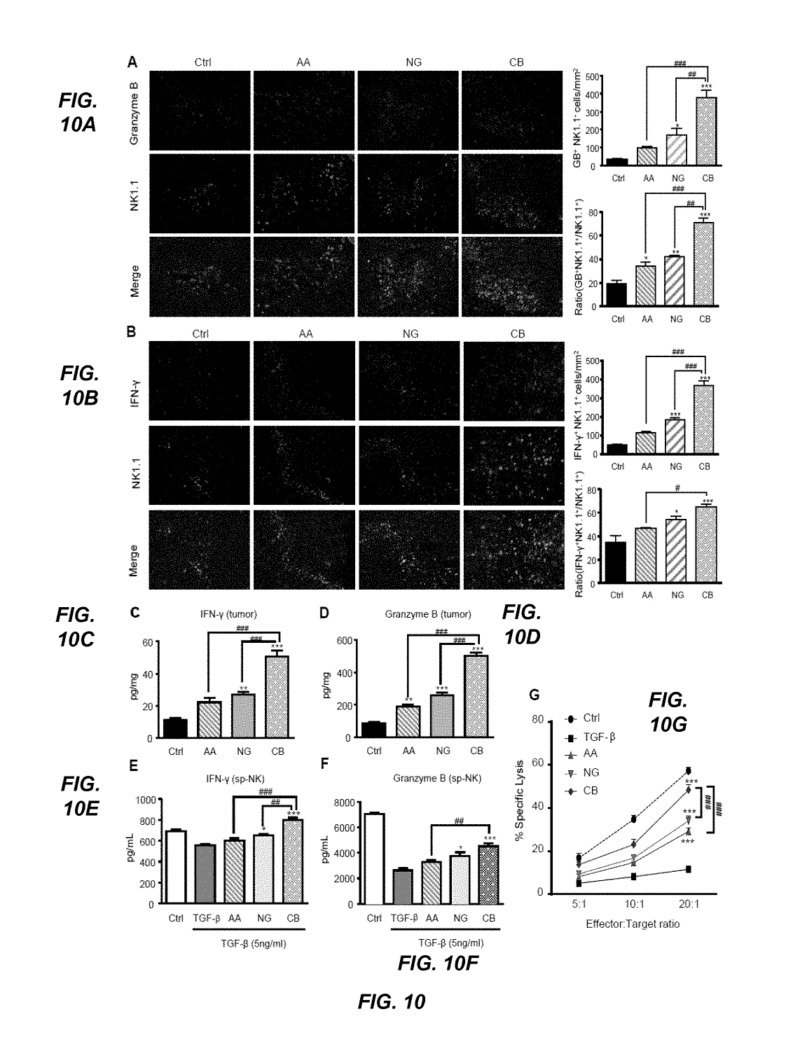

A safe dosage of HLPC-purified naringenin (NG) and asiatic acid (AA) was first determined in normal mice by peritoneal injection with different dosages of AA or NG for toxicity assays and a dose of AA at 10 mg/kg body weight and NG at 50 mg//Kg body weight is selected for further examination of the therapeutic efficacy on cancer (FIG. 1). The effects of combined use of a safe dosage of naringenin (NG, 10 mg/kg) and asiatic acid (AA, 50 mg/kg) at a ratio of AA:NG=5:1 was selected and the therapeutic effect on cancer was tested in two syngeneic mouse models of invasive lung carcinoma (LLC) and melanoma (B16F10). Unexpectedly, it was found that compared with AA or NG single treatment, the combination therapy (AA+NG) showed a synergistic effect on inhibition of tumor progression, including the tumor growth rate and tumor weight (FIG. 2), without causing extra toxicity (FIG. 3). It was also identified that the therapeutic mechanisms whereby the combination therapy largely promote both NK cell differentiation and cancer-killing activities in the tumor microenvironment (FIGS. 4, 5), inhibits tumor growth/proliferation (Ki67+) by blocking angiogenesis (CD31+) and vascular endothelial growth factor (VEGF) expression (FIG. 6), and suppresses tumor invasive activities such as the expression of matrix metalloproteinases (MMPs), including MMP-2, MMP-9 and MMP-13 (FIG. 7) and tumor migration activities as demonstrated by the wound healing and transwell assays (FIG. 8). Thus, the combination of NG and AA presents as a new emerging and promising therapy for tumor.

### Example 2 Combination of Asiatic Acid and Naringenin Inhibits Tumor Progression by Enhancing the NK Cell Immunity Against Cancer

TGF-β1 plays a promoting role in tumor growth, invasion and metastasis via a mechanism associated with hyperactive Smad3 signaling while suppressing Smad7 in tumor microenvironment. The present study revealed that rebalancing Smad3/Smad7 signaling with asiatic acid (AA, a Smad7 agonist) and naringenin (NG, a Smad3 inhibitor) can significantly inhibit tumor progression in two syngeneic mouse tumor models of invasive melanoma (B16F10) and lung carcinoma (LLC). It was discovered that compared to the monotherapy, combination therapy with AA and NG produced a synergistic effect on inhibiting tumor growth on both B16F10 and LLC models. Mechanistically, it was discovered that Smad3 physically bound transcriptional factors of Id2 and IRF2 to suppress NK cell development and functions. Treatment with AA and NG greatly inactivated Smad3 while restoring Smad7 signaling, therefore largely promoting NK cell differentiation, maturation and cytotoxicity against cancer via the Id2/IRF2-dependent mechanism. This was further confirmed by disrupting Id2 or IRF2 to blunt the protective effect of AA and NG on NK cell development. In conclusion, treatment with AA and NG produces a synergistic effect on inactivating TGF-β/Smad3 signaling and therefore suppresses melanoma and lung carcinoma growth by promoting Id2/IRF2-dependent NK cell immunity against cancer. Thus, combinational therapy with AA and NG may represent a novel, safe and surprisingly effective anti-cancer therapy for clinical application.

### Introduction

Due to the genetic heterogeneity of cancer and the less-specificity of cytotoxic drugs against cancer, increasing evidence shows that immunotherapy by targeting tumor microenvironment may be a promising approach for anti-cancer therapy (1). Among the signaling molecules in tumor microenvironment, TGF-β1 signaling has been proved to be a tumor promoter in tumor growth, invasion, and metastasis (2-5). Therefore, altering tumor microenvironment from supportive to inhibitory effect on cancer by targeting TGF-β1 signaling may represent a prospective therapeutic target.

As a vital transcriptional factor in TGF-β1 downstream signaling, Smad3 is essential for tumor progression (6). Previous studies have confirmed that enhanced expression of Smad3 is observed in human colorectal cancer (7), while mice lacking Smad3 were resistant to chemical-induced skin carcinogenesis (8, 9). A recent study also revealed that TGF-β1/Smad3 signaling is essential for cancer growth and invasion via a mechanism associated with suppressing NK cell immunity against cancer in tumor microenvironment (10). On the contrary, as an inhibitory Smad protein, Smad7 inhibits the phosphorylation of Smad2 and Smad3 via a negative feedback loop, thus preventing the over-activation of TGF-β1/Smad3 signaling (11, 12). Low expression level of Smad7 has been reported to correlate with poor prognosis and lymph node metastasis in pancreatic cancer (13). Meanwhile, overexpression of Smad7 inhibits primary tumor growth and metastasis and produces a better clinical outcome in several cancer models (14, 15). Thus, it was hypothesized that rebalancing Smad3 and Smad7 signaling in tumor microenvironment may be a novel therapeutic strategy for cancer, which was examined in this study.

NK cells play a pivotal role in tumor immunosurveillance, which can rapidly respond to tumor formation and inhibit tumor progression independent from antigen presentation and T cell immunity (16-18). However, NK cell-mediated cytotoxicity on tumor cells is blunted by TGF-β1 in tumor microenvironment (19). On one hand, TGF-β1 is responsible for NK cell immaturity during mouse infancy through inhibiting the expression of E4BP4, T-bet and GATA-3, which are crucial for NK cell differentiation and terminal maturation (10, 20). On the other hand, TGF-β1 markedly impairs NK cell-mediated cytotoxicity via reducing cytokine productions of NK cells, such as granzymes and IFN-γ (19, 21). As mentioned above, overexpression of Smad3 and loss of Smad7 are responsible for TGF-β1 induced tumor progression. Thereby it was postulated that retrieving the balance between Smad3 and Smad7 may strengthen NK cell-mediated immune response in TGF-β1 rich tumor microenvironment by enhancing NK cell maturation and restoring NK cell cytotoxicity against cancer.

Asiatic acid (AA), a triterpene from Centella asiatica, has been shown to have anti-inflammation, antioxidation, neuroprotection, and promoting wound healing (22). One recent study also reported that AA can function as a Smad7 agonist capable of inducing Smad7 to alleviate TGF-β1-induced liver fibrosis (23). In contrast, naringenin (NG), a natural predominant flavanone isolated of citrus, functions as an effective inhibitor for Smad3 in liver and pulmonary fibrosis (24, 25). Interestingly, combination of AA and NG can effectively treat renal fibrosis in the unilateral ureteral obstruction (UUO) model by synergistically inhibiting Smad3 while promoting Smad7 signaling (26). Thus, it has been hypothesized that the combination of AA and NG may enhance the therapeutic effect on cancer by retrieving the balance between Smad3 and Smad7 signaling in tumor microenvironment. This was tested by treating two syngeneic mouse tumor models, melanoma (B16F10) and lung carcinoma (LLC), with combination of AA and NG. The therapeutic effects and inhibitory mechanisms of AA and NG on tumor progression were investigated.

### Results

### Combination Therapy of AA and NG Synergistically Inhibits Melanoma and Lung Carcinoma Growth in Syngeneic Model Models

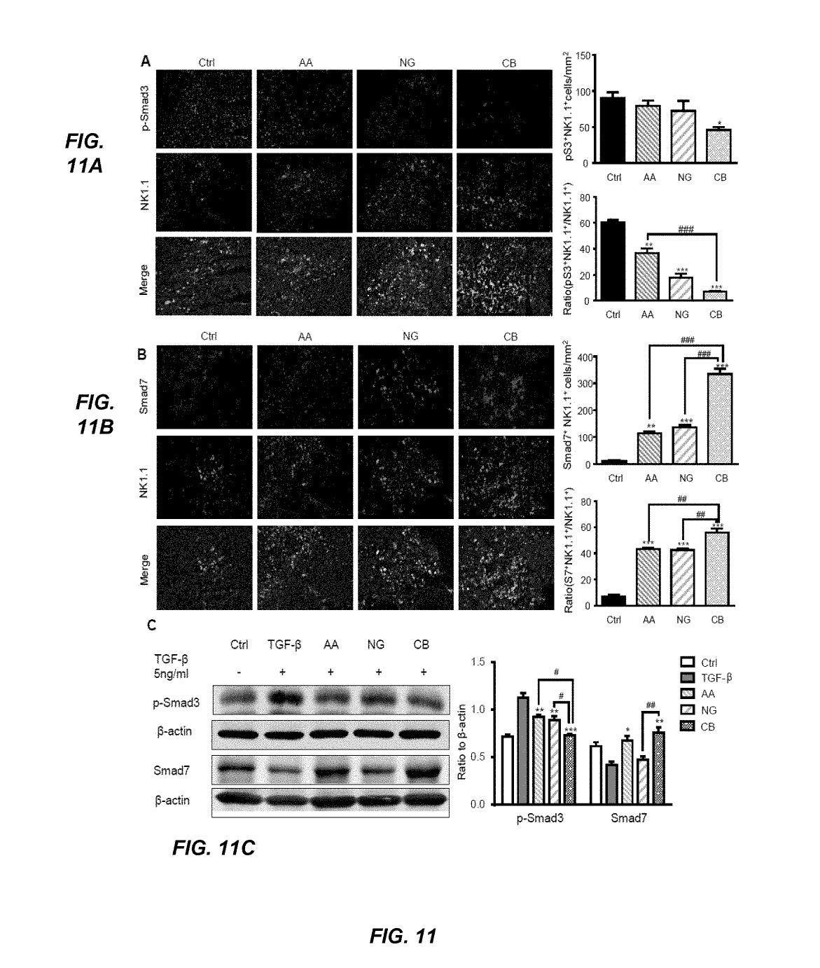

It was first determined an effective dosage of AA or NG on B16F10 melanoma mouse model. As shown in FIG. 1(A-D), single treatment with either 10 mg/kg AA or 50 mg/kg NG effectively inhibited the melanoma progression, although no further significant improvement on tumor growth was found on higher dosages of AA or NG. Therefore, AA at 10 mg/kg and NG at 50 mg/kg were selected as optimal dosages for the combination therapy in the present study. Results shown in FIG. 1 (E-F) clearly demonstrated that the combined treatment with AA and NG synergistically restrained tumor volume since day 10 after tumor inoculation when compared with the monotherapy of AA or NG. The anti-tumor efficacy of combination therapy was further validated on LLC lung cancer mouse model, where a better anti-tumor effect on the tumor growth rate, bioluminescence imaging, and tumor weights over the monotherapy were achieved (FIG. 11G-J).

In addition, it was also found that combination therapy with AA(10 mg/kg) and NG(50 mg/kg) did not induce significant bone marrow suppression (FIG. 19A) and the toxicity to the kidney, heart, and liver on LLC-bearing mice, as the count of white blood cells were not significantly decreased and the serum creatinine, LDH, AST and ALT were not significantly increased in mice receiving combination therapy compared with normal control or single AA or NG-treated mice (FIG. 19B-E).

### Combination Therapy Enhances NK Cell Immune Response in Tumor Microenvironment

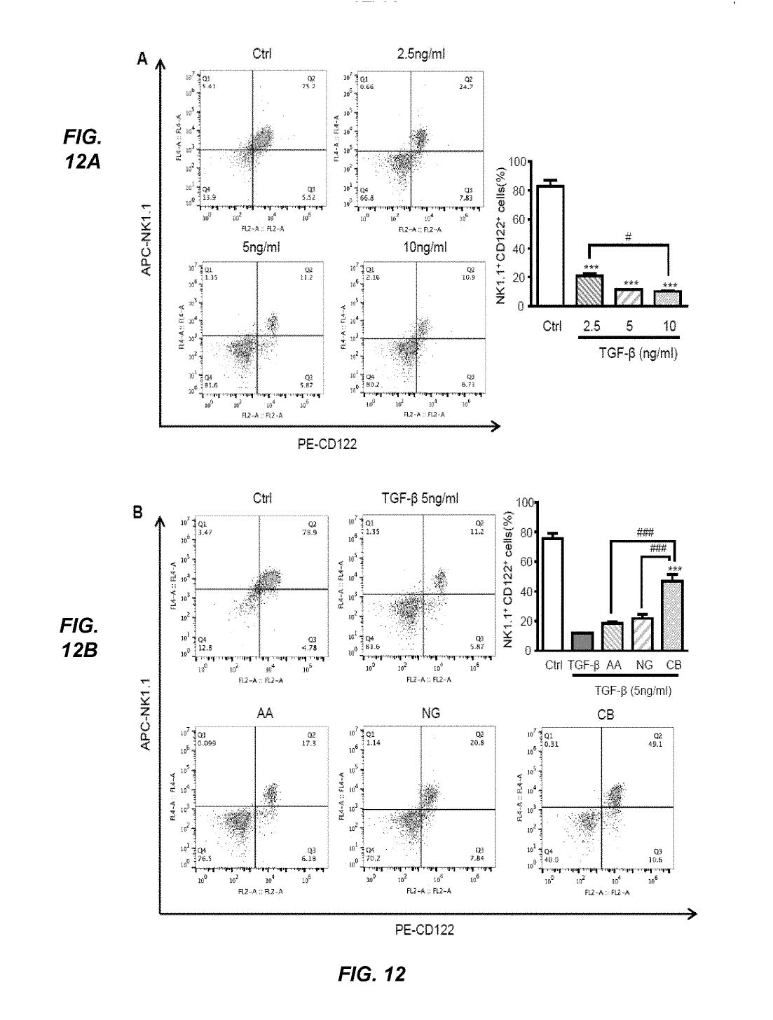

It was recently found that tumor-infiltrating NK cells were markedly decreased in TGF-β1 rich tumor microenvironment of LLC lung carcinoma in a mouse model (10). As shown in FIG. 12A, compared to the single treatment with AA or NG, combination therapy more effectively increased tumor-infiltrating cytotoxic NK cells labeled with NK1.1 and NKp46, suggesting that treatment with AA and NG largely promotes cytotoxic NK cell population locally in tumor microenvironment. Similarly, combination therapy with AA and NG also significantly promoted systemic NK cell response in peripheral blood as determined by two-color flow cytometry (FIG. 12B). These findings revealed that the therapeutic efficacy of AA and NG on tumor may attribute to promoting NK cell anti-tumor activities by accelerating the NK cell development and homing to tumor microenvironment. However, as shown in FIG. 20, combination therapy with AA and NG did not influence the proliferative activity of NK cells and expression of CXCR3, a prerequisite for NK cell infiltration (27).

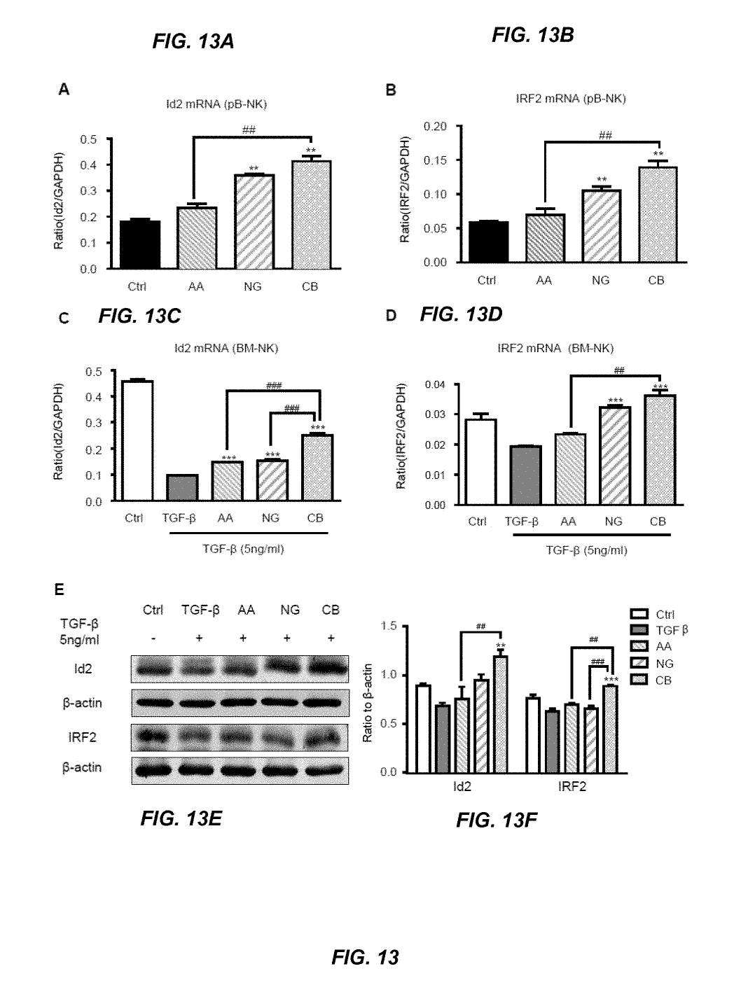

It is well established that TGF-β1 markedly impairs NK cell-mediated cytotoxicity via inhibiting various cytokine productions including granzyme B (GB) and IFN-γ (19). It was also detected that treatment with AA or NG significantly increased granzyme B-secreting NK cells (GB⁺ NK1.1⁺ cells) and IFN-γ-producing NK cells (IFN-γ⁺ NK1.1⁺ cells) in the tumor microenvironment, which were further enhanced in LLC tumor-bearing mice receiving combination therapy (FIG. 13A, B). Meanwhile, ELISA also detected a markedly increase in IFN-γ and granzyme B in tumor tissue of mice treated with combination therapy when compared with those treated with control solvent and monotherapy with AA or NG (FIG. 13C, D). This observation was further verified on cultured splenic NK cells treated with either AA or NG, or AA plus NG under high TGF-β1 conditions (FIG. 13E, F).

It was next assessed whether the combination therapy enhances the tumor-killing capacity of NK cells by co-culturing splenic NK cells with B16F10 melanoma cells at ratio of 5:1, 10:1 and 20:1. As results shown in FIG. 13G, single treatment with AA or NG increased NK cell-mediated cytotoxicity against melanoma cells in a dose-dependent manner, which was further enhanced by the combination therapy.

Combination Therapy with AA and NG Attenuates the Inhibitory Effect of TGF-β1 on NK Cell Development Via Rebalancing Smad3 and Smad7 Signaling

Next, a further hypothesis that combination therapy with AA and NG facilitates NK cell development by rebalancing Smad3 and Smad7 was tested in tumor microenvironment. As expected, treatment with AA and NG dramatically blocked phosphorylation of Smad3 (p-Smad3) while boosted Smad7 expression in tumor infiltrated NK cells compared with monotherapy on LLC lung carcinoma-bearing mice. More importantly, while the proportion of p-Smad3⁺ NK1.1⁺ cells was markedly reduced, the proportion of Smad7⁺ NK1.1⁺ cells was considerably increased in the tumor microenvironment treated with AA and NG when compared to mice treated with control or monotherapy. These results indicate the competence of combination therapy to rebalance Smad3 and Smad7 signaling in tumor infiltrated NK cells (FIG. 14A, B).

To further explore the mechanism of combination therapy facilitating NK cell immunity against cancer in TGF-β rich microenvironment, 10 μM of AA and 100 μM of NG (according to the molar ratio in vivo) were applied on bone marrow-derived NK cells in vitro. As show in FIG. 14C, addition of TGF-β1 increased the phosphorylation level of Smad3, which was attenuated by AA or NG but was synergistically suppressed by their combination treatment. Meanwhile, AA and NG monotherapy were capable of restoring TGF-β1-induced suppression of Smad7 expression, whereas, combination therapy reversed the inhibitory effect of TGF-β1 on Smad7 expression (FIG. 14C).

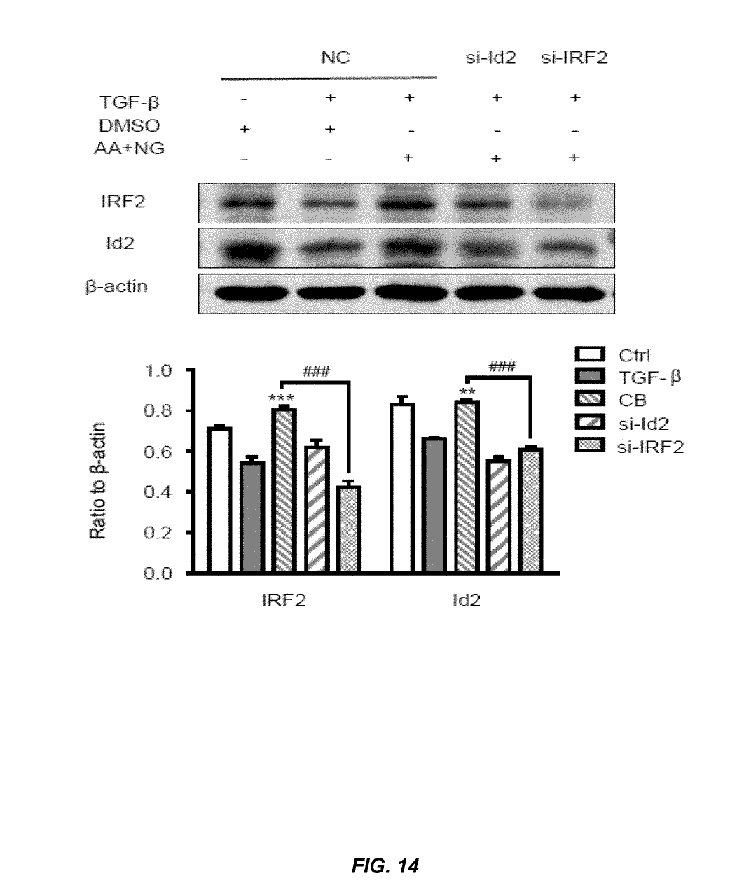

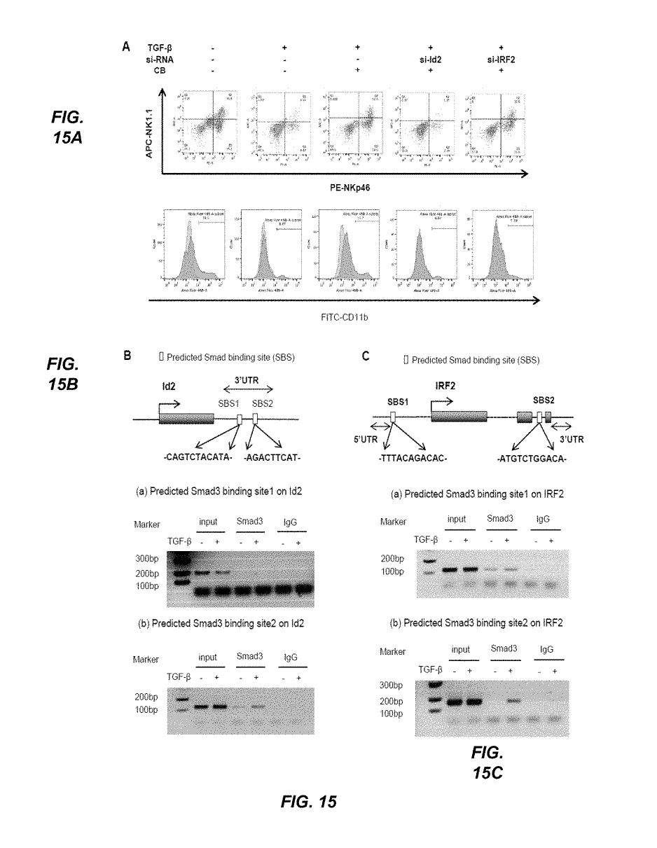

As increased NK cell population in peripheral blood and tumor microenvironment in AA and NG treated LLC mice was not associated with the proliferation or recruitment of NK cells (FIG. 20), it was speculated that the combination therapy may facilitate NK cell immunity against cancer by promoting NK cell development in tumor bearing mice. To verify this hypothesis, the influence of combination therapy on bone marrow-derived NK cell development in vitro was examined. As shown in FIG. 15A, addition of TGF-β1 significantly suppressed the NK cell development in a dose-dependent manner. For example, TGF-β1 at a dose of 5 ng/ml largely reduced NK1.1⁺ CD122⁺ cell (immature NK cells) proportion from 80% to 11%. Addition of AA or NG moderately attenuated this inhibitory effect, which was markedly enhanced by the combination treatment with AA and NG (FIG. 15B).

Combination Therapy with AA and NG Promotes NK Cell Development Via Id2 and IRF2-Dependent Mechanisms

It is known that NK cell differentiation and maturation are sterically regulated by various transcription factors including Id2 and IRF2 (28, 29). It was then examined whether enhanced NK cell maturation by AA and NG is transcriptionally regulated by Id2 and IRF2. Real-time PCR detected that expression of Id2 and IRF2 were significantly enhanced in NK cells isolated from peripheral blood of LLC lung carcinoma-bearing mice that received combination therapy with AA and NG when compared with control or monotherapy (FIG. 16A, B). This in vivo observation was further confirmed in vitro in bone marrow-derived NK cells that TGF-β1-induced suppression of Id2 and IRF2 in NK cells was significantly reduced by a single dose of AA or NG but was blunted by their combination treatment (FIG. 16C-E). Therefore it was speculated that combination therapy may enhance NK cell maturation in TGF-β1-rich tumor microenvironment through restoring the expression of Id2 and IRF2, two essential transcription factors respectively responsible for NK cell lineage commitment and NK cell terminal maturation (30, 31). This was further confirmed by silencing Id2 on NK cells to impair the protective effect of AA and NG on NK1.1+NKp46+ population (mature NK cells) under high TGF-β1 condition (FIG. 21 and FIG. 17A, C). Surprisingly, silencing of IRF2 gave no significant influence on NK cell development in response to AA and NG, but reduced terminal maturation of NK cells as demonstrated by expression of CD11b (FIG. 17). This is consistent with the previous report that IRF2 is a checkpoint regulator during the process of NK cell terminal maturation (31). Therefore, combination of AA and NG therapy promoted NK cell development in an Id2/IRF2 pathway in TGF-β1-rich tumor microenvironment.

The regulatory mechanism of TGF-β/Smad signaling in Id2/IRF2-dependent NK cell differentiation and maturation was next examined, and predicted Smad3 binding sites (SBS) were found nearby IRF2 3′UTR and 5′UTR, and another two on Id2 3′UTR with ECR browser (FIG. 18A,B). ChIP assay confirmed that addition of TGF-β1 markedly enhanced the physical binding of Smad3 protein on the genomic sequences of Id2 3′UTR and IRF2 nearby 3′UTR (FIG. 18C, D), thereby suppressing the transcription of Id2 and IRF2 in NK cells. This finding demonstrates a direct regulatory mechanism of TGF-β/Smad3 on transcriptionally inhibition of Id2/IRF2-dependent NK cell development.

### Discussion

Traditional Chinese Medicine (TCM) has been used in Chinese health care for thousands of years. The principle of TCM is to regain the balance between the “Yin-Yang” and maintain the homeostasis of human body with various kinds of herbs or natural products. As a holistic medicine, herbs used in TCM are generally assembled according to formula, which is called “Fu-Fang” in Chinese, to achieve an optimized efficacy thorough the interaction within the herbs and to reduce the side effect of certain medicinal herbs. Accumulating evidence demonstrates that the imbalance between Smad3 and Smad7 plays a vital role in a variety of pathological conditions including tissue fibrosis and tumor microenvironment (10,26). In the present study, the inventor provided the first evidence for rebalancing the TGF-β/Smad signaling in tumor microenvironment by a combination therapy with AA (a Smad7 agonist) and NG (a Smad3 specific inhibitor) to synergistically inhibit tumor progression in mouse models of melanoma and lung carcinoma.

The most significant finding in the present study is not only the first evidence for a combination therapy with AA and NG to synergistically inhibit tumor growth in both melanoma and lung carcinoma, but also a clear mechanism by which the combined AA and NG therapy suppressed cancer progression by promoting the NK cell immunity against tumors through rebalancing TGF-β/Smad signaling in tumor microenvironment. Compared to previous studies of AA or NG on tumor cell cytotoxicity (32-34), the present study added new therapeutic mechanism through which combination of AA and NG suppressed tumor growth by switching Smad3-dependent immunosuppressive tumor microenvironment to immunoreactive one as evidenced by largely increasing both NK. cell number (NK1.1+CD122+ and NK1.1+NKp46+) and anti-cancer activities such as production of IFN-γ and granzyme B. This finding is consistent with previous report that genetic disruption of Smad3 or pharmacological inhibition of Smad3 protects against cancer progression in mouse models of melanoma and lung carcinoma (10).

Here the present inventor also identified that inhibition of Smad3-Id2/IRF2-mediated immunosuppression on NK cell development and functions may be an essential mechanism whereby combination therapy with AA and NG effectively inhibited cancer progression. As an indispensable component in tumor immunosurveillance, NK cells exert rapid innate immune response to tumorigenesis and cancer progression by directly triggering tumor cell death (16, 18). However, NK cell immune response has been severely suppressed by hyperactive TGF-β1/Smad signaling in tumor microenvironment characterized by markedly decreased NK cell accumulation and loss of NK cell cytotoxicity against cancer (20, 35). Given the capability of rebalancing Smad3 and Smad7 signaling, combination of AA and NG may serve as a potent therapeutic strategy to restore the NK cell immunity against cancer in TGF-β1-abundant tumor microenvironment.

It is well established that NK cell development is strictly programmed by a number of transcription factors and TGF-β1 inhibits CD11b^{high}CD43^{high} NK cells via suppressing both T-bet and GATA3 (20). One recent study also identified that TGF-β1 via Smad3 promotes cancer progression by inhibiting E4BP4-mediated NK cell development (10). In the present study, it was also identified that combination treatment with AA and NG promoted Id2/IRF2-dependent NK cell differentiation and maturation via rebalancing the Smad3/Smad7 signaling. Id2, as the antagonist of E proteins, is indispensable for the development of NK cell precursor to immature NK cells, and also involved in NK maturation (30, 36, 37); meanwhile, IRF2 protects premature NK cells from apoptosis to ensure NK cells completing GATA3-induced terminal maturation as well as maintaining NK cell proliferation (31, 38). Interestingly, it was discovered that TGF-β1 not only hampered NK cell terminal maturation by reducing CD11b′ NK cell proportion via suppressing IRF2 signaling, but also impaired the production of immature NK via blocking Id2 dependent NK cell lineage commitment. Moreover, silencing Id2 or IRF2 in bone marrow-derived NK cells consequently impaired the rescuing effect of combination therapy, respectively on the production of immature NK cells or on terminal maturation of CD11^{high} Dx5^{high} stage in TGF-β1 environment. This in-depth study confirmed that Smad3 inhibited Id2 and IRF2 via directly interacting with their 3′UTR as a transcription repressor. Thereby, it was verified that combination therapy with AA and NG increased NK cell production in tumor bearing mice via alleviating the inhibition of TGF-β1 on Id2 and IRF2-dependent NK cell differentiation and maturation both in vivo and in vitro.

Previously, Massagué et al. identified that TGF-β1 activates Smad2/3 together with ATF1 transcription factors to directly bind to the promoter region of both interferon-γ and granzyme B to induce a significant reduction in cytokine productions (39, 40). Thereby, suppressing hyperactive TGF-β1/Smad signaling via rebalancing Smad3 and Smad7 signaling by combination therapy debilitated the inhibition of TGF-β1 on IFN-γ and granzyme B production in NK cells as noted in this study. Cytotoxicity assay ultimately confirmed combination therapy with AA and NG not only promoted the production of cytokines, but effectively elevated NK cell cytotoxicity against tumor cells compared with either AA or NG monotherapy.

In summary, the present study provides a new, effective and safe TCM-based therapy for melanoma and lung carcinoma in mice with combination of AA and NG. It also identifies the underlying mechanism through which combined treatment with AA and NG synergistically promoted NK cell immunity against cancer by suppressing TGF-β/Smad3-mediated inhibition of Id2/IRF2-dependent NK cell differentiation and maturation. Thus, treatment with AA and NG can be a promising TCM-based therapeutic strategy for cancer clinically.

### Materials and Methods

### Syngeneic Mouse Tumor Model and Treatment

B16F10 melanoma syngeneic tumor model was generated in C57/Bl6 male mice (aged week 8 and 25 g body weight) via subcutaneous injection of 1×10⁶ B16F10 cells. Lewis Lung Cancer syngeneic tumor model was also generated in C57/Bl6 male mice via subcutaneous injection of 2×10⁶ luciferase-labeled LLC cells. When average tumor size reached 50 mm³, mice were randomly divided into four groups respectively receiving drug administration as described below. Tumor progression was monitored by calculating tumor volume with vernier calipers as well as by bioluminescence imaging with IVIS Spectrum system (Xenogen, PerkinElmer, Inc., MA, USA). All animal experiments were carried out by a protocol approved by Animal Ethics Experimental Committee at the Chinese University of Hong Kong.

Two TCM-based drugs were used in this study, including asiatic acid (95% HLPC-purified, Nanning, China) and naringenin (98% HLPC-purified, Xian, China). Both drugs were dissolved in DMSO and Tween-80 at the ratio of 9:1, which was further diluted 10 times with saline as working solution. Tumor-bearing mice were randomly divided into groups for various treatments with AA at a dose of 10 mg/kg body weight, NG at a dose of 50 mg/kg body weight, or combination therapy with AA (10 mg/kg) and NG (50 mg/kg) every day via intraperitoneal injection. Studies were carried out by a protocol approved by Animal Ethics Experimental Committee at the Chinese University of Hong Kong.

### Immunofluorescence Staining

Immunofluorescence staining was performed with 5 μm PLP fixed frozen sections of mouse tumor tissue to detect NK cell infiltration in tumor microenvironment and the protein levels of p-Smad3, Smad7, IFN-γ and granzyme B in these tumor infiltrated NK cells. Primary antibodies used for immunofluorescence staining include Alexa488 conjugated anti-mouse NK-1.1 (Biolegend, CA, USA), PE conjugated anti-mouse NKp46 (eBioscience Inc., CA, USA), anti-p-Smad2/3 (Santa Cruz Biotechnology, Santa Cruz, Calif.), anti-Smad7, anti-interferon gamma and anti-granzyme B (Abcam, MA, USA). All slides were mounted with DAPI containing mounting medium then analyzed with fluorescence microscope (Leica Microsystems, Wetzlar, Germany).

### NK Cells Culture In Vitro

After euthanasia, bone marrow cells were isolated from C57BL/6 mice following the protocol as previously described (10). Bone marrow cells were then seeded at the density of 1×10⁶ cells/ml in MEMα medium (Gibco, Thermo Fisher Scientific Inc., MA, USA) containing 10% FBS supplemented with 1 ng/ml IL-7, 10 ng/ml Flt3L, 30 ng/ml SCF, 50 ng/ml IL-15 (Peprotech, NJ, USA) and 50 mM β-Mercaptoethanol (Gibco, Thermo Fisher Scientific Inc., MA, USA) and cultured for 4 days. Then transferred cells to MEMα medium containing 10% FBS supplemented with 50 ng/ml IL-15, 20 ng/ml IL-2 and 50 mM β-Mercaptoethanol to induce NK cells maturation for another 3 days.

To isolate splenic NK cells, spleen tissues were gently mashed through a 40 μm cell strainer into a 50 mm² dish and then splenic NK cells were isolated with NK Cell Isolation Kit II (Miltenyi Biotec Inc., CA, USA). Splenic NK cells were cultured in MEMα medium containing 10% FBS supplemented with 50 ng/ml IL-15 and 20 ng/ml IL-2.

siRNA Transfection

Id2 and IRF2 were knocked down on freshly isolated mouse bone marrow cells via transfecting siRNA specific for IRF2 (5′-GCAAGCAGUACCUCAGCAATT-3′), siRNA specific for Id2 (5′-GCACGTCATCGATTACATC-3′) and nonsense control (5′-UUCUCCGAACGUGUCACGUTT-3′) on day 0 and day 4 with Lipofectamine RNAiMAX transfection system (Life technologies, NY, USA) and Opti-MEM medium (Gibco, Thermo Fisher Scientific Inc., MA, USA) and cells were collected on day 6 for flow cytometry analysis.

### Cytotoxicity Assay

B16F10 melanoma cells were harvested as target cells and bone marrow-derived NK cells treated with AA or NG for 24 hours were harvested as effector cells. Cells were thoroughly washed and seeded in 96-well plates at the effector: target (E:T) ratios of 5:1, 10:1 and 20:1. Incubated in 5% CO2 incubator at 37° C. for 6 hours then detect the LDH release of each well with CytoTox 96® Non-Radioactive Cytotoxicity Assay kit (Promega Corporation, WI, USA). Read absorbance at 490 nm and calculated the percentage of NK cell mediated cytotoxicity with the following formula.

$$Cytotoxicity = \frac{Experimental - Effector Spontaneous - Target Spontaneous}{Target Maximum - Target Spontaneous}$$

### Flow Cytometry Analyses

Peripheral blood NK cells and cultured NK cells were analyzed using various markers as previously described (10), including rat anti-mouse CD16/CD32 antibody (BD Pharmingen, BD Biosciences, CA, USA), APC-conjugated anti-mouse NK1.1, PE-conjugated anti-mouse NKp46, PE-conjugated anti-mouse CD122 (eBioscience Inc., CA, USA) and Alexa 488-conjugated anti-mouse/human CD11b Antibody (Biolegend, CA, USA). After immunostaining, cells were resuspended with 200 μl staining buffer and analyzed with BD Accuri™ C6 Cytometer (BD Biosciences, CA, USA).

### ELISA

Tumor homogenates and supernatants from cultured NK cells were measured for IFN-γ and granzyme B levels by using enzyme-linked immunosorbent assay (ELISA) with Mouse IFN-gamma ELISA Kit (R&D Systems, Inc. MN, USA) and Mouse Granzyme B ELISA Kit (eBioscience Inc., CA, USA).

### ChIP Assay

Bone marrow-derived NK cells were stimulated with 5 ng/ml TGF-β1 for 1 hour and collected by centrifuge at 2000 rpm for 5 minutes. After fixed by cross-linking with 37% formaldehyde, total chromatin was isolate with SimpleChIP® Enzymatic Chromatin IP Kit (Cell signaling, MA, USA) as instructed by manufacturer. Cross-linked Smad3-DNA complexes were precipitated with Smad3 (C67H9) antibody (Cell signaling, MA, USA) and normal anti-rabbit IgG (Cell signaling, MA, USA). Targeted genomic regions were subsequently detected by PCR with specific primers for predicted conserved Smad binding site listed in Table 2 and analyzed with gel electrophoresis.

### RNA Extraction and Real-Time PCR

Total RNA was isolated from tumor tissue or cultured cells with PureLink® RNA Mini Kit (Life technologies, NY, USA) according to manufacturer's instructions. RNA concentration was measured with Spectrophotometers Nanodrop (ND-2000, Thermo Fisher Scientific Inc., MA, USA). Real-time PCR program was performed with Bio-Rad iQ SYBR Green supermix with Opticon2 (Bio-Rad, Hercules, Calif., USA) according to manufacturer's instructions on CFX96 Touch™ Real-Time PCR Detection System (Bio-Rad, Hercules, Calif., USA). GAPDH was used as the internal control and the ratio of target to GAPDH was calculated as ΔCt=Ct (target)−Ct (GAPDH), Ratio (target)=2(^{-ΔCt}). Primers used for Real-Time PCR were listed in Table 1.

### Western Blot

Tumor tissues and cultured NK cells were lysed with ice-cold RIPA buffer, and then protein was isolated as previously described (2 6). Membranes were blocked with 5% BSA in PBS for 1 hour at room temperature before incubated with primary antibodies against Smad7, Id2, β-actin (Santa Cruz Biotechnology, Santa Cruz, Calif., USA), p-Smad3 and IRF2 (Cell signaling, MA, USA) overnight at 4° C., subsequently incubated with IRDye 800-conjugated secondary antibodies (Rockland immunochemicals, PA, USA). Target protein expression was then detected by LiCor/Odyssey infrared image system (LI-COR Biosciences, NE, USA) and analyzed with Image J software (NIH, Bethesda, Md., USA).

### Statistical Analyses

Data were presented as mean±stand error of mean (SEM). All data were analyzed with GraphPad Prism 6.0 software (San Diego, Calif., USA) by one-way ANOVA for single variable analysis or two-way ANOVA for two independent variables analysis, followed by Tukey's multiple comparisons test.

All patents, patent applications, and other publications, including GenBank Accession Numbers, cited in this application are incorporated by reference in the entirety for all purposes.

TABLE 1
Sequence of primers for real-time PCR
Primers for Real-time PCR

| Target | | |
|---|---|---|
| Gene | Forward Primer | Reverse Primer |
| Id2 | ACCAGAGACCTGGACAGAAC | AAGCTCAGAAGGGAATTCAG |
| IRF2 | CTTATCCGAACGACCTTCCA | CTTGCTGTCCAGATGGGACT |
| CXCR3 | TGCTAGATGCCTCGGACTTT | CGCTGACTCAGTAGCACAATT |
| GAPDH | GCATGGCCTTCCGTGTTC | GATGTCATCATACTTGGCAGGTTT |

TABLE 2
Sequence of primers for ChIP assay.
Primers for ChIP Assay
Target

| Gene | Forward Primer | Reverse Primer |
|---|---|---|
| Id2 SBS1 | GGGGTGAGAGAACAGAAGGA | TTTCAGACAACCAGTGCTTTG |
| Id2 SBS2 | CAGCATTCAGTAGGCTCGTG | GCCTTTTCACAAAGGTGGAG |
| IRF2 | GGTGTCGTGTGTTGTGGGTA | GGTGGCGACAGTGTCTGTAA |
| 5′UTR | | |
| SBS1 | | |
| IRF2 | GTGTCTCAGCTCCACCCATT | CTCCTATGCTCAGCCTGTCC |
| 3′UTR | | |
| SBS2 | | |

### LIST OF REFERENCES

1. J. A. Joyce, Therapeutic targeting of the tumor microenvironment. Cancer Cell 7, 513-520 (2005).
2. J. A. Joyce, D. T. Fearon, T cell exclusion, immune privilege, and the tumor microenvironment. Science 348, 74-80 (2015).
3. J. Massague, TGFβ in Cancer. Cell 134, 215-230 (2008).
4. S. B. Jakowlew, Transforming growth factor-beta in cancer and metastasis. Cancer Metastasis Rev 25, 435-457 (2006).
5. L. Yang, Y. Pang, H. L. Moses, TGF-beta and immune cells: an important regulatory axis in the tumor microenvironment and progression. Trends Immunol 31, 220-227 (2010).
6. C.-H. Heldin, K. Miyazono, P. ten Dijke, TGF-[beta] signalling from cell membrane to nucleus through SMAD proteins. Nature 390, 465-471 (1997).
7. O. Korchynskyi, M. Landström, R. Stoika, K. Funa, C. H. Heldin, P. ten Dijke, S. Souchelnytskyi, Expression of Smad proteins in human colorectal cancer. International journal of cancer 82, 197-202 (1999).
8. A. G. Li, S.-L. Lu, M.-X. Zhang, C. Deng, X.-J. Wang, Smad3 knockout mice exhibit a resistance to skin chemical carcinogenesis. Cancer research 64, 7836-7845 (2004).
9. Q. Fan, D. Gu, H. Liu, L. Yang, X. Zhang, M. C. Yoder, M. H. Kaplan, J. Xie, Defective TGF-β Signaling in Bone Marrow-Derived Cells Prevents Hedgehog-Induced Skin Tumors. Cancer research 74, 471-483 (2014).
10. P. M.-K. Tang, S. Zhou, X.-M. Meng, Q.-M. Wang, C.-J. Li, G-Y Lian, X.-R. Huang, Y-J. Tang, X.-Y. Guan, B. P.-Y. Yan, Smad3 promotes cancer progression by inhibiting E4BP4-mediated NK cell development. Nature Communications 8, 14677 (2017).
11. A. Moustakas, S. Souchelnytskyi, C.-H. Heldin, Smad regulation in TGF-β signal transduction. Journal of Cell Science 114, 4359-4369 (2001).
12. R. Derynck, Y. E. Zhang, Smad-dependent and Smad-independent pathways in TGF-β family signalling. Nature 425, 577-584 (2003).
13. P. Wang, J. Fan, Z. Chen, Z.-Q. Meng, J.-M. Luo, J.-H. Lin, Z.-H. Zhou, H. Chen, K. Wang, Z.-D. Xu, Low-level expression of Smad7 correlates with lymph node metastasis and poor prognosis in patients with pancreatic cancer. Annals of surgical oncology 16, 826-835 (2009).
14. A. Lamora, J. Talbot, G. Bougras, J. Amiaud, M. Leduc, J. Chesneau, J. Taurelle, V. Stresing, M. C. Le Deley, M. F. Heymann, Overexpression of smad7 blocks primary tumor growth and lung metastasis development in osteosarcoma. Clinical Cancer Research 20, 5097-5112 (2014).
15. T. Feng, J. Dzieran, X. Gu, S. Marhenke, A. Vogel, K. Machida, T. S. Weiss, P. Ruemmele, O. Kollmar, P. Hoffmann, Smad7 regulates compensatory hepatocyte proliferation in damaged mouse liver and positively relates to better clinical outcome in human hepatocellular carcinoma. Clinical Science 128, 761-774 (2015).
16. E. Vivier, D. H. Raulet, A. Moretta, M. A. Caligiuri, L. Zitvogel, L. L. Lanier, W. M. Yokoyama, S. Ugolini, Innate or adaptive immunity? The example of natural killer cells. Science 331, 44-49 (2011).
17. A. E. Wakil, Z. E. Wang, J. C. Ryan, D. J. Fowell, R. M. Locksley, Interferon gamma derived from CD4(+) T cells is sufficient to mediate T helper cell type 1 development. J Exp Med 188, 1651-1656 (1998).
18. I. Waldhauer, A. Steinle, NK cells and cancer immunosurveillance. Oncogene 27, 5932-5943 (2008).
19. G. Bellone, M. Aste-Amezaga, G. Trinchieri, U. Rodeck, Regulation of NK cell functions by TGF-beta 1. The Journal of Immunology 155, 1066-1073 (1995).
20. J. P. Marcoe, J. R. Lim, K. L. Schaubert, N. Fodil-Cornu, M. Matka, A. L. McCubbrey, A. R. Farr, S. M. Vidal, Y. Laouar, TGF-beta is responsible for NK cell immaturity during ontogeny and increased susceptibility to infection during mouse infancy. Nat Immunol 13, 843-850 (2012).
21. Y. Laouar, F. S. Sutterwala, L. Gorelik, R. A. Flavell, Transforming growth factor-beta controls T helper type 1 cell development through regulation of natural killer cell interferon-gamma. Nat Immunol 6, 600-607 (2005).
22. D. Bian, J. Zhang, X. Wu, Y Dou, Y. Yang, Q. Tan, Y. Xia, Z. Gong, Y Dai, Asiatic acid isolated from Centella asiatica inhibits TGF-beta1-induced collagen expression in human keloid fibroblasts via PPAR-gamma activation. International journal of biological sciences 9, 1032-1042 (2013).
23. L.-x. Tang, R.-h. He, G. Yang, J.-j. Tan, L. Zhou, X.-m. Meng, X. R. Huang, H. Y Lan, Asiatic acid inhibits liver fibrosis by blocking TGF-beta/Smad signaling in vivo and in vitro. PLoS One 7, e31350 (2012).
24. X. Liu, W. Wang, H. Hu, N. Tang, C. Zhang, W. Liang, M. Wang, Smad3 specific inhibitor, naringenin, decreases the expression of extracellular matrix induced by TGF-β1 in cultured rat hepatic stellate cells. Pharmaceutical research 23, 82-89 (2006).
25. G. Du, L. Jin, X. Han, Z. Song, H. Zhang, W. Liang, Naringenin: a potential immunomodulator for inhibiting lung fibrosis and metastasis. Cancer research 69, 3205-3212 (2009).
26. X.-m. Meng, Y. Zhang, X.-R. Huang, G-l. Ren, J. Li, H. Y Lan, Treatment of renal fibrosis by rebalancing TGF-β/Smad signaling with the combination of asiatic acid and naringenin. Oncotarget 6, 36984 (2015).
27. M. Wendel, I. E. Galani, E. Suri-Payer, A. Cerwenka, Natural killer cell accumulation in tumors is dependent on IFN-gamma and CXCR3 ligands. Cancer Res 68, 8437-8445 (2008).
28. A. Marçais, S. Viel, M. Grau, T. Henry, J. Marvel, T. Walzer, Regulation of mouse NK cell development and function by cytokines. Frontiers in immunology 4, 450 (2013).
29. J. P. Di Santo, Natural killer cell developmental pathways: a question of balance. Annu Rev Immunol 24, 257-286 (2006).
30. T. Ikawa, S. Fujimoto, H. Kawamoto, Y. Katsura, Y. Yokota, Commitment to natural killer cells requires the helix-loop-helix inhibitor Id2. Proceedings of the National Academy of Sciences 98, 5164-5169 (2001).
31. S. Taki, S. Nakajima, E. Ichikawa, T. Saito, S. Hida, IFN regulatory factor-2 deficiency revealed a novel checkpoint critical for the generation of peripheral NK cells. The Journal of Immunology 174, 6005-6012 (2005).
32. M. S. I. Abaza, K. Y. Orabi, E. Al-Quattan, R. a. J. Al-Attiyah, Growth inhibitory and chemo-sensitization effects of naringenin, a natural flavanone purified from Thymus vulgaris, on human breast and colorectal cancer. Cancer Cell International 15, 46 (2015).
33. X.-L. Tang, X.-Y Yang, H.-J. Jung, S.-Y Kim, S.-Y Jung, D.-Y Choi, W.-C. Park, H. Park, Asiatic acid induces colon cancer cell growth inhibition and apoptosis through mitochondrial death cascade. Biological and Pharmaceutical Bulletin 32, 1399-1405 (2009).
34. C. Lou, F. Zhang, M. Yang, J. Zhao, W. Zeng, X. Fang, Y Zhang, C. Zhang, W. Liang, Naringenin decreases invasiveness and metastasis by inhibiting TGF-β-induced epithelial to mesenchymal transition in pancreatic cancer cells. PLoS One 7, e50956 (2012).
35. H.-G Kopp, T. Placke, H. R. Salih, Platelet-derived transforming growth factor-β down-regulates NKG2D thereby inhibiting natural killer cell antitumor reactivity. Cancer research 69, 7775-7783 (2009).
36. M. D. Boos, Y Yokota, G. Eberl, B. L. Kee, Mature natural killer cell and lymphoid tissue-inducing cell development requires Id2-mediated suppression of E protein activity. The Journal of experimental medicine 204, 1119-1130 (2007).
37. Y. Yokota, A. Mansouri, S. Mori, S. Sugawara, S. Adachi, S.-I. Nishikawa, P. Gruss, Development of peripheral lymphoid organs and natural killer cells depends on the helix-loop-helix inhibitor Id2. Nature 397, 702-706 (1999).
38. M. Lohoff, G. S. Duncan, D. Ferrick, H.-W. Mittrucker, S. Bischof, S. Prechtl, M. Röllinghoff, E. Schmitt, A. Pahl, T. W. Mak, Deficiency in the transcription factor interferon regulatory factor (IRF)-2 leads to severely compromised development of natural killer and T helper type 1 cells. The Journal of experimental medicine 192, 325-336 (2000).
39. D. A. Thomas, J. Massagué, TGF-β directly targets cytotoxic T cell functions during tumor evasion of immune surveillance. Cancer cell 8, 369-380 (2005).
40. J. Yu, M. Wei, B. Becknell, R. Trotta, S. Liu, Z. Boyd, M. S. Jaung, B. W. Blaser, J. Sun, D. M. Benson, Pro- and antiinflammatory cytokine signaling: reciprocal antagonism regulates interferon-gamma production by human natural killer cells. Immunity 24, 575-590 (2006).

What is claimed is:

1. A method for inhibiting proliferation of a cell, comprising the step of contacting the cell with an effective amount of naringenin and Asiatic acid.

2. The method of claim 1, wherein the cell is a cancer cell.

3. The method of claim 1, wherein the cell is a melanoma or lung cancer cell.

4. The method of claim 1, wherein the cell is within a person's body.

5. The method of claim 4, wherein the contacting step comprises subcutaneous, intramuscular, intravenous, intraperitoneal, or oral administration.

6. The method of claim 4, wherein the effective amount is about 5 mg/kg to 250 mg/kg body weight of naringenin and 1 mg/kg to 50 mg/kg body weight of Asiatic acid.

7. The method of claim 1, wherein naringenin and Asiatic acid are administered at a weight ratio of about 10:1 to about 1:10, preferably about 1:5.

8. The method of claim 1, wherein naringenin and Asiatic acid are administered in a single composition.

9. The method of claim 1, wherein naringenin and Asiatic acid are administered in two separate compositions.

10. The method of claim 1, wherein naringenin and Asiatic acid are administered in the form of a solution, a powder, a gel, a cream/paste, a tablet, or a capsule.

11. A composition comprising (1) an effective amount of naringenin and Asiatic acid and (2) a pharmaceutically acceptable excipient.

12. The composition of claim 11, wherein naringenin and Asiatic acid are present in the weight ratio of about 10:1 to about 1:10, preferably about 1:5.

13. The composition of claim 11, which is formulated for subcutaneous, intramuscular, intravenous, intraperitoneal, topical, or oral administration.

14. The composition of claim 11, which is a solution, a powder, a gel, a cream/paste, a tablet, or a capsule.

15. A kit for inhibiting cell proliferation, comprising a first container containing a first composition that comprises narigenin and a second container containing a second composition that comprises Asiatic acid.

16. The kit of claim 15, wherein the first or second composition is formulated for subcutaneous, intramuscular, intravenous, intraperitoneal, topical, or oral administration.

17. The kit of claim 12, further comprising an instruction manual for administration of the first and second compositions.


SEQUENCE LISTING (22 sequences)

SEQ ID NO: 1 - DNA, 21 bp, Artificial Sequence
  Synthetic construct
gcaagcaguaccucagcaatt

SEQ ID NO: 2 - DNA, 19 bp, Artificial Sequence
  Synthetic construct
gcacgtcatcgattacatc

SEQ ID NO: 3 - DNA, 21 bp, Artificial Sequence
  Synthetic construct
uucuccgaacgugucacgutt

SEQ ID NO: 4 - DNA, 20 bp, Artificial Sequence
  Synthetic construct
accagagacctggacagaac

SEQ ID NO: 5 - DNA, 20 bp, Artificial Sequence
  Synthetic construct
aagctcagaagggaattcag

SEQ ID NO: 6 - DNA, 20 bp, Artificial Sequence
  Synthetic construct
cttatccgaacgaccttcca

SEQ ID NO: 7 - DNA, 20 bp, Artificial Sequence
  Synthetic construct
cttgctgtccagatgggact

SEQ ID NO: 8 - DNA, 20 bp, Artificial Sequence
  Synthetic construct
tgctagatgcctcggacttt

SEQ ID NO: 9 - DNA, 21 bp, Artificial Sequence
  Synthetic construct
cgctgactcagtagcacaatt

SEQ ID NO: 10 - DNA, 18 bp, Artificial Sequence
  Synthetic construct
gcatggccttccgtgttc

SEQ ID NO: 11 - DNA, 24 bp, Artificial Sequence
  Synthetic construct
gatgtcatcatacttggcaggttt

SEQ ID NO: 12 - DNA, 20 bp, Artificial Sequence
  Synthetic construct
ggggtgagagaacagaagga

SEQ ID NO: 13 - DNA, 21 bp, Artificial Sequence
  Synthetic construct
tttcagacaaccagtgctttg

SEQ ID NO: 14 - DNA, 20 bp, Artificial Sequence
  Synthetic construct
cagcattcagtaggctcgtg

SEQ ID NO: 15 - DNA, 20 bp, Artificial Sequence
  Synthetic construct
gccttttcacaaaggtggag

SEQ ID NO: 16 - DNA, 20 bp, Artificial Sequence
  Synthetic construct
ggtgtcgtgtgttgtgggta

SEQ ID NO: 17 - DNA, 20 bp, Artificial Sequence
  Synthetic construct
ggtggcgacagtgtctgtaa

SEQ ID NO: 18 - DNA, 20 bp, Artificial Sequence
  Synthetic construct
gtgtctcagctccacccatt

SEQ ID NO: 19 - DNA, 20 bp, Artificial Sequence
  Synthetic construct
ctcctatgctcagcctgtcc

SEQ ID NO: 20 - DNA, 11 bp, Artificial Sequence
  Synthetic construct
cagtctacata

SEQ ID NO: 21 - DNA, 11 bp, Artificial Sequence
  Synthetic construct
tttacagacac

SEQ ID NO: 22 - DNA, 11 bp, Artificial Sequence
  Synthetic construct
atgtctggaca